SEQUENCE LISTING (17 sequences)

SEQ ID NO: 1 - protein, 376 aa, Herpes simplex virus
MASYPGHQHASAFDQAARSRGHSNRRTALRPRRQQEATEVRPEQKMPTLLRVYIDGPHGMGKTTTTQLLVALGSRDDIVYVPEPMTYWRVLGASETIANIYTTQHRLDQGEISAGDAAVVMTSAQITMGMPYAVTDAVLAPHIGGEAGSSHAPPPALTLIFDRHPIAALLCYPAARYLMGSMTPQAVLAFVALIPPTLPGTNIVLGALPEDRHIDRLAKRQRPGERLDLAMLAAIRRVYGLLANTVRYLQCGGSWREDWGQLSGTAVPPQGAEPQSNAGPRPHIGDTLFTLFRAPELLAPNGDLYNVFAWALDVLAKRLRSMHVFILDYDQSPAGCRDALLQLTSGMVQTHVTTPGSIPTICDLARTFAREMGEAN

SEQ ID NO: 2 - protein, 250 aa, Drosophila melanogaster
MAEAASCARKGTKYAEGTQPFTVLIEGNIGSGKTTYLNHFEKYKNDICLLTEPVEKWRNVNGVNLLELMYKDPKKWAMPFQSYVTLTMLQSHTAPTNKKLKIMERSIFSARYCFVENMRRNGSLEQGMYNTLEEWYKFIEESIHVQADLIIYLRTSPEVAYERIRQRARSEESCVPLKYLQELHELHEDWLIHQRRPQSCKVLVLDADLNLENIGTEYQRSESSIFDAISSNQQPSPVLVSPSKRQRVAR

SEQ ID NO: 3 - protein, 234 aa, Lycopersicon esculentum
MAFSSSARNPVDLRNGSKNSFCPVGEIHVIVGPMFAGKTTALLRRVNLESNDGRNVVLIKSSKDARYAVDAVVTHDGTRFPCWSLPDLSSFKQRFGKDAYEKVDVIGIDEAQFFGDLYEFCCNAADFDGKIIVVAGLDGDYLRKSFGSVLDIIPLADTVTKLTARCELCNRRAFFTFRKTNETETELIGGADIYMPVCRQHYVNGQSVNESAKMVLESHKVSNELILESPLVDP

SEQ ID NO: 4 - protein, 361 aa, Arabidopsis thaliana
MVDYLRSSVGIIHRNHAESITTFIKESVDDELKDSGPEPNLNVKKRLTFCVEGNISVGKSTFLQRIANETVELQDLVEIVPEPVDKWQDVGPDHFNILDAFYSEPQRYAYTFQNYVFVTRLMQEKESASGVKPLRLMERSVFSDRMVFVRAVHEAKWMNEMEISIYDSWFDPVVSSLPGLVPDGFIYLRASPDTCHKRMMLRKRAEEGGVSLKYLQDLHEKHESWLLPFESGNHGVLSVSRPSLHMDNSLHPDIKDRVFYLEGNHMHSSIQKVPALVLDCEPNIDFSRDIEAKTQYARQVAEFFEFVKKKQETSTEKSNSQSPVLLPHQNGGLWMGPAGNHVPGLDLPPLDLKSLLTRPSA

SEQ ID NO: 5 - protein, 250 aa, Drosophila melanogaster
MAEAASCARKGTKYAEGTQPFTVLIEGNIGSGKTTYLNHFEKYKNDICLLTEPVEKWRNVNGVNLLELMYKDPKKWAMPFQSYATLTMLQSHTAPTNKKLKIMERSIFSARYCFVENMRRNGSLEQGMYNTLEEWYKFIEESIHVQADLIIYLRTSPEVAYERIRQRARSEESCVPLKYLQELHELHEDWLIHQRRPQSCKVLVLDADLDLENIGTEYQRSESSIFDAISSNQQPSPVPVSPSKRQRVAR

SEQ ID NO: 6 - protein, 580 aa, Arabidopsis thaliana
MQKILCKSTTSSTPVLSTPVNSLAAGFISLGFKTPVKNLPPCSTTKPLSTCFFSTSAMPTTTASVSSGGVGFSAYLQRTVHKPAPASVRFSTAGYRTCRCSIDGTNRAWVGRTGSWRALFCSDSTGGLTPVNATAGAVVESEEESDGEDEDEEKDEKPVRMNRRNRSSSGSGEFVGNPDLLKIPGVGLRNQRKLVDNGIGDVAELKKLYKDKFWKASQKMVDYLRSSVGIIHRNHAESITTFIKESVDDELKDSGPEPNLNVKKRLTFCVEGNISVGKSTFLQRIANETVELQDLVEIVPEPVDKWQDVGPDHFNILDAFYSEPQRYAYTFQNYVFVTRLMQEKESASGVKPLRLMERSVFSDRMVFVRAVHEAKWMNEMEISIYDSWFDPVVSSLPGLVPDGFIYLRASPDTCHKRMMLRKRAEEGGVSLKYLQDLHEKHESWLLPFESGNHGVLSVSRPSLHMDNSLHPDIKDRVFYLEGNHMHSSIQKVPALVLDCEPNIDFSRDIEAKTQYARQVAEFFEFVKKKQETSTEKSNSQSPVLLPHQNGGLWMGPAGNHVPGLDLPPLDLKSLLTRPSA

SEQ ID NO: 7 - protein, 300 aa, Oryza sativa
MVEFLQSSVGIIHKNHAESITLFIKESVDEELKGTDSPNVSKNKRLTFCVEGNISVGKTTFLQRIANETIELRDLVEIVPEPIAKWQDVGPDHFNILDAFYAEPQRYAYTFQNYVFVTRVMQEKESSSGIKPLRLMERSVFSDRMVVKFLKVFVRAVHEANWMNEMEISIYDSWFDPVVSSLPGLIPDGFIYLRASPDTCHKRMMVRKRSEEGGVTLDYLRGLHEKHESWLLPSKGQGPGVLSVSQVPVHMEGSLPPDIRERVFYLEGDHMHSSIQKVPALVLDCEHDIDFNKDIEAKRQ

SEQ ID NO: 8 - protein, 260 aa, Homo sapiens [sequence omitted]

SEQ ID NO: 9 - protein, 277 aa, Homo sapiens [sequence omitted]

SEQ ID NO: 10 - protein, 234 aa, Homo sapiens
MGAFCQRPSSDKEQEKEKKSVICVEGNIAGGKTTCLEFFSNATDVEVLTEPVSKWRNVRGHNPLGLMYHDASRWGLTLQTYVQLTMLDRHTRPQVSSVRLMERSIHSARYIFVENLYRSGKMPEVDYVVLSEWFDWILRNMDVSVDLIVYLRTNPETCYQRLKKRCREEEKVIPLEYLEAIHHLHEEWLIKGSLFPMAAPVLVIEADHHMERMLELFEQNRDRILTPENRKHCP

SEQ ID NO: 11 - protein, 234 aa, Homo sapiens
MSCINLPTVLPGSPSKTRGQIQVILGPMFSGKSTELMRRVRRFQIAQYKCLVIKYAKDTRYSSSFCTHDRNTMEALPACLLRDVAQEALGVAVIGIDEGQFFPDIMEFCEAMANAGKTVIVAALDGTFQRKPFGAILNLVPLAESVVKLTAVCMECFREAAYTKRLGTEKEVEVIGGADKYHSVCRLCYFKKASGQPAGPDNKENCPVPGKPGEAVAARKLFAPQQILQCSPAN

SEQ ID NO: 12 - protein, 248 aa, Bombyx mori
MSANNVKPFTVFVEGNIGSGKTTFLEHFRQFEDITLLTEPVEMWRDLKGCNLLELMYKDPEKWAMTFQSYVSLTMLDMHRRPAPTPVKLMERSLFSARYCFVEHIMRNNTLHPAQFAVLDEWFRFIQHNIPIDADLIVYLKTSPSIVYQRIKKRARSEEQCVPLSYIEELHRLHEDWLINRIHAECPAPVLVLDADLDLSQITDEYKRSEHQILRKAVNVVMSSPNKHSPKKPISTTPIKITPHMRIL

SEQ ID NO: 13 - protein, 246 aa, Anopheles gambiae
MPPIASEKLGASGKKPFTVFVEGNIGSGKTTFLNHFQKFNDICLLTEPVEKWRNCGGVNLLDLMYKESHRWAMPFQTYVTLTMLDMHTCQTDKSVKLMERSLFSARNCFVESMLASGSLHQGMYNVLQEWYDFICCNIHIQADLIVYLQTSPEVVYERMKQRARSEESCVPLEYLKELHELHENWLIHGASPRPAPVLVLNADLDLNTIGAEYERSETSILKPILIENTNQHAILTSPAKRAKTDF

SEQ ID NO: 14 - protein, 276 aa, Oryza sativa
MSSICAMRSLLAASTFLRSGASPLLRPLSRPLPSRLNLSRFGPVRPVSAAAAAADKSRGGGGSAMEAQPSYPGEIHVIVGPMFAGKTTALLRRVQVEAGTGRNVALIKSDKDNRYGLDSVVTHDGTKMPCWALPELSSFQDKLGTEAYDKVDVIGIDEAQFFDDLHDFCCKAADRDGKIVVVAGLDGDYKRNKFGSVLDIIPLADSVTKLTARCELCGRRAFFTLRKTRETKTELIGGADVYMPVCRQHYLDGQIVIEATRIVLDLEKSKVIHAFK

SEQ ID NO: 15 - protein, 238 aa, Arabidopsis thaliana
MATLKASFLIKTLDSDVTGDFLSDLERRGSGAVHVIMGPMFSGKSTSLLRRIKSEISDGRSVAMLKSSKDTRYAKDSVVTHDGIGFPCWALPDLMSFPEKFGLDAYNKLDVIGIDEAQFFGDLYEFCCKVADDDGKIVIVAGLDGDYLRRSFGAVLDIIPIADSVTKLTARCEVCGHKAFFTLRKNCDTRTELIGGADVYMPVCRKHYITNHIVIKASKKVLEDSDKARAESCVAATI

SEQ ID NO: 16 - protein, 277 aa, Arabidopsis thaliana
MRTLISPSLAPFSLHLHKPSLFSTALRFSFSINNITPTNSPPSTISTRKLQTKATRVTSSSSSQPLSSSSPGEIHVVVGPMFSGKTTTLLRRILAERETGKRIAIIKSNKDTRYCTESIVTHDGEKYPCWSLPDLSSFKERFGFDDYENRLDVIGIDEAQFFGDLYEFCREAADKEGKTVIVAGLDGDFMRRRFGSVLDLIPIADTVTKLTSRCEVCGKRALFTMRKTEEKETELIGGAEVYMPVCRSHYVCGQNVLETARAVLDSSNNHSVVASSL

SEQ ID NO: 17 - protein, 365 aa, Lycopersicon esculentum
MVEFLQSSIGIIHRNHAESITTYIRKSVDEELKENNSDSNVKSTQKKRLTFCVEGNISVGKTTFLQRIANETLELQDLVEIVPEPIAKWQDIGPDHFNILDAFYAEPQRYAYTFQNYVFVTRVMQERESSGGIRPLRLMERSVFSDRMVFVRAVHEANWMNEMEISIYDSWFDPVVSTLPGLIPDGFIYLRASPDTCHKRMMLRKRTEEGGVSLEYLRGLHEKHESWLFPFESGNHGVLSVSELPLNFDKFCVPPEIRDRVFYLEGNHMHPSIQKVPALVLDCEPNIDFNRDIEAKRQYARQVADFFEFVKKKQEVMPGAGEEQPKGNQAPVMLPQNGGLWVPGGKFSESTLNLDFRRNMSFMSH

(19) United States
(12) Patent Application Publication
(10) Pub. No.: US 2007/0264241 A1
(43) Pub. Date: Nov. 15, 2007

(54) Compounds for Enhanced Cancer Therapy

(76) Inventors: Tomas J. Ekstrom, Huddinge (SE); Per M. Almqvist, Bromma (SE); Thomas Asklund, Stockholm (SE)

Correspondence Address: BIRCH STEWART KOLASCH & BIRCH, PO BOX 747, FALLS CHURCH, VA, 22040-0747, US

(21) Appl. No.: 10/588,379

(22) PCT Filed: Feb. 25, 2005
(86) PCT No.: PCT/EP05/50805

§ 371 Date: Aug. 2, 2006

Related U.S. Application Data
(60) Provisional application No. 60/547,058, filed on Feb. 25, 2004.

(30) Foreign Application Priority Data
Feb. 25, 2004 (DK) PA 2004 00302

(51) Int. Cl. A61K 48/00; A61K 31/19

(52) U.S. Cl. 424/93.21; 424/94.5; 514/44; 514/45; 514/568; 514/7

(57) ABSTRACT

The present invention relates to methods and compositions for enhanced cancer treatment based on nucleoside analogue prodrugs. In a preferred embodiment, the invention relates to enhanced suicide cancer therapy.

The present application claims the benefit of U.S. provisional patent application No. 60/547,058 filed on 25 Feb. 2004, which is incorporated by reference in its entirety. It claims priority from Danish patent application No. PA 2004 00302 filed on 25 Feb. 2004. All references cited in those applications and in the present application are hereby incorporated by reference in their entirety.

### FIELD OF THE INVENTION

The present invention relates to methods and compositions for enhanced cancer treatment based on nucleoside analogue prodrugs. In a preferred embodiment, the invention relates to enhanced suicide cancer therapy.

### BACKGROUND

Tumour cells modified to express a Thymidine Kinase (TK) gene acquire the ability to convert the non-toxic nucleoside analog ganciclovir (GCV) to its cytotoxic metabolite ganciclovir-triphosphate. Cells genetically engineered to express this “suicide” gene are eliminated if exposed to ganciclovir. Experimental tissue culture of tumour cells as well as brain tumour implants, consisting of a mixture of TK-expressing cells and unmodified “native” tumour cells also regress following ganciclovir treatment without harm to adjacent normal tissue. This phenomenon, where a minority of TK-expressing cells lead to the death and elimination of adjacent native tumour cells not expressing TK, has been termed the “bystander effect”. Therapy based on delivery of nucleoside analogues such as AZT alone (where the drug is activated by cells' own thymidine kinases) also depends on the bystander effect.

The “bystander” effect is dependent on cell-cell contacts and on intercellular communication mediated by gap junctions. Gap junctions are proteinaceous channels connecting cells and allow passage of small molecules and ions up to approximately 1000 Da. Gap junctions can mediate transfer of phosphorylated ganciclovir from TK-positive to TK-negative tumour cells.

Phagocytosis of gancidovir-phosphate laden cell debris by adjacent tumour cells also leads to cell death. Blood vessel endothelial cells within or adjacent to the tumour may also acquire TK, and their destruction with ganciclovir therapy may also contribute to tumour regression. “Suicide” tumour cells release inflammatory cytokines which promote hemorrhagic necrosis in local, but non-contiguous, tumour deposits. Furthermore, tumours undergoing a necrotic death, as opposed to apoptotic cell death, will up-regulate the expression of proteins such as hsp70, IL10 and IL12, which may enhance immune recognition and rejection. Necrotic tumours may be infiltrated with a wide assortment of immunocompetent cells such as CD4+ lymphocytes, CD8+ lymphocytes, NK cells and Antigen Presenting Cells.

These infiltrating cells may take part in a tumour-specific immune response, which is an important component of the local as well as distant anti-tumour immune bystander effect (Moolten, F. L., Cancer Research, 46: 5276-5281, 1986).

Intracerebral tumours are also susceptible to immune clearance following suicide gene expression, suggesting that the brain is not an immune sanctuary for cancer.

Malignant brain tumours are an appealing target for suicide gene delivery, since the entire malignancy is confined to the brain and amenable to eradication by the bystander effect. Key components for the success of this strategy are the genetic vector from which the suicide gene is expressed and its delivery vehicle.

Glioblastoma multiforme (GBM) is a form of brain cancer, which is lethal to 50% of afflicted patients within 12 months and 90% of afflicted patients within 24 months. The therapeutic resistance of GBM is related to its invasiveness and cellular heterogeneity (Misra A, Chattopadhyay P, Dinda A K, Sarkar C, Mahapatra A K, Hasnain S E, et al. Extensive intra-tumor heterogeneity in primary human glial tumors is a result of locus non-specific genomic alterations. (J Neurooncol 2000; 48(1):1-12)) and tumor invasiveness to the exceptional migratory nature of tumour cells with their ability to diffusely infiltrate normal brain tissue (Lipinski C A, Tran N L, Bay C, Kloss J, McDonough W S, Beaudry C, et al. Differential role of proline-rich tyrosine kinase 2 and focal adhesion kinase in determining glioblastoma migration and proliferation. Mol Cancer Res 2003; 1(5):323-32). In parallel with pre-clinical research and clinical trials of various anti-neoplastic agents, it is crucial to explore new therapeutic paradigms, since the standard treatment armamentarium of surgery, radiation and chemotherapy only give minor beneficial effects on overall survival, as reported (Stewart L A. Chemotherapy in adult high-grade glioma: a systematic review and meta-analysis of individual patient data from 12 randomised trials. Lancet 2002; 359(9311):1011-8).

One main obstacle for efficient suicide gene therapy is the difficulty of reaching a sufficient proportion of cells with the therapeutic gene or gene product. One way to address this issue in vitro is to enhance the bystander effect by transfection with the structural gap junction protein connexin 43 (Cx43) gene It is known that recombinant expression of connexin proteins enhance the bystander effect in suicide gene therapy in vitro (Estin D, Li M, Spray D, Wu J K, “Connexins are expressed in primary brain tumors and enhance the bystander effect in gene therapy”. Neurosurgery 1999; 44(2):361-8; discussion 368-9) and in vivo (Dilber M S, Abedi M R; Christensson B, Bjorkstrand B, Kidder G M, Naus C C, et al. Gap junctions promote the bystander effect of herpes simplex virus thymidine kinase in vivo. Cancer Res 1997; 57(8):1523-8), presumably by up-regulation of gap junction communication. In a clinical setting recombinant expression of connexin proteins is not a feasible way of treating cancer. Other ways of improving the therapeutic efficacy of TK based cancer therapy comprise more efficient and targeted delivery of the gene therapy vector to the cancer cells and/or by selecting and/or developing TK genes coding for enzymes with improved kinetic properties over HSV-TK (see e.g. WO 00/36099 “New medical use of gene and vector encoding a multisubstrate deoxyribonucleoside kinase (dNK)”, WO 01/88106 “Multi-substrate insect deoxynucleoside kinase variants”, and WO 03/100045 “Plant thymidine kinases (TK)”).

These references, however do not address the problem of achieving sufficient activation of nucleoside analogue prodrugs in a sufficient proportion of the cancer cells to obtain killing of the whole cancer tumour.

### SUMMARY OF THE INVENTION

In a first aspect the invention relates to a pharmaceutical composition comprising at least one compound capable of enhancing gap-junction communication and at least one nucleoside analogue. By providing this composition, the therapeutic efficacy of the nucleoside analogue can be enhanced by the enhanced transfer of activated nucleoside analogues between cells via gap-junctions. The composition also provides enhanced selectivity for cancer tumours. The nucleoside analogues are selectively toxic to dividing cells, and by adding the enhancer of gap-junction communication, the selectivity for cancer tumours, which comprise numerous gap-junctions, is increased.

The invention is based on the novel finding by the present inventors that gap-junction communication, which is very important for several kinds of cancer therapy, in particular thymidine kinase based cancer therapy, can be enhanced by simple small organic molecules, which are relatively non-toxic to human beings, exemplified by the aromatic fatty acid 4-phenylbutyrate.

In a preferred embodiment, the composition further comprises a source of a deoxyribonudeoside kinase capable of activating the nucleoside analogue to a cytotoxic drug. Through this combination, both the efficacy and the selectivity of the treatment can be improved. The selectivity can be improved as the kinase can be delivered locally by the use of gene therapy or by liposome-mediated delivery targeted for cancer cells. The efficacy can be improved since the targeted cells express larger amounts of kinase, and since kinases with improved kinetic properties can be used.

In a further aspect, the invention relates to a method of treating cancer by administering to a patient a therapeutically effective amount of at least one compound capable of increasing gap-junction communication, and at least one nucleoside analogue. Preferably, the method further comprises administration of a source of deoxyribonucleoside kinase.

In a further aspect the invention relates to the use of at least one compound capable of enhancing gap-junction communication, and at least one nucleoside analogue, for the preparation of a medicament for the treatment of cancer.

Preferably, the medicament further comprises a source of deoxyribonucleoside kinase.

In a further aspect the invention relates to a method of augmenting the therapeutic activity of a nucleoside analogue based cancer therapy, said method comprising administering to a patient an amount of at least one compound capable of enhancing gap-junction communication and thereby augmenting the therapeutic activity of said nucleoside analogue based therapy.

Preferably the nucleoside analogue based therapy further comprises administration of a source of deoxyribonucleoside kinase.

In a still further aspect the invention relates to pharmaceutical articles containing at least one nucleoside analogue and at least one compound capable of enhancing gap-junction communication as a combination for the simultaneous, separate or successive administration in cancer therapy.

Preferably the articles further comprise a source of deoxyribonudeoside kinase.

### FIGURES

FIG. 1. Antiproliferative effect of 4-phenylbutyrate (4-PB) on glioblastoma cultures. A. The cell cultures were treated with 4-PB at indicated concentrations and for indicated times, after which they were analyzed by the MTT test as described in the Methods section. B-D. Phase contrast micrographs of glioblastoma culture (hGBM-5) non-treated (B), treated for 24 hours (C), and for 10 days (D), at a concentration of 5 mM 4-PB. Statistical significance between means was assessed by Student's t-test for unpaired values. ***P<0.001 relative to control untreated cells (n=4).

FIG. 2. Concentration-dependent effects of compounds on cell viability shown bv MTT test. Glioblastoma hGBM-1 (♦), hGBM-5 (□) and hGBM-14 (▪) cells were cultured with valproic acid (A), splitomycin (B), sodium butyrate (C) and TSA (D) for 24 hours. Data are expressed as a percentage of untreated control cells ±SE, n=4. Statistical significance between means was assessed by Student's t-test for unpaired values, *p<0.05; **p<0.01, ***p<0.001, relative to control untreated hGBM-14 cells; ^{#}p<0.05; ##p<0.01, ^{###}p<0.001, relative to control untreated hGBM-1 cells and ^{§}p<0.05; ^{§§}p<0.01, ^{§§§}p<0.001, relative to control untreated hGBM-5 cells.

FIG. 3. Immunocytochemical analysis of GFAP expression in primary human glioblastoma cells. Glioblastoma cultures hGBM-1, hGBM-5 and hGBM-14 were immunostained for GFAP following treatment of 5 mM 4-PB for 48 hours (D-F), compared to control cultures (A-C). Note the morphological differentiation of glioblastoma cells following 4-PB treatment as seen by GFAP immunofluorescence as well as in insets of phase contrast images. GFAP immunodetection is increased in parallel to the redistribution of GFAP to a nuclear/perinuclear localisation in addition to the usual cytoplasmic distribution.

FIG. 4. Immunodetection of GFAP in human glioblastoma primary cultures by Western blot analysis in the presence and absence of 4-PB. Glioblastoma cells cultured in the presence and absence of 4-phenylbutyrate, were recovered and processed for Western blot analysis of GFAP expression in protein extracts (60 μg/lane). Anti-GFAP positive proteins correspond to non-phosphorylated (P0) and phosphorylated (P) species of GFAP. (A), (B) and (C) show the relative content of P0 and P forms of GFAP in the abscense and in the presence of increasing concentrations of 4-PB in hGBM-1, hGBM-5 and hGBM-14, respectively. Note the increase of non-phosphorylated forms of GFAP in hGBM-5 and hGBM-14 when treated with 4-PB. Statistical significance between means was assessed by Student's t-test for unpaired values. *P<0.05 relative to control untreated cells (n=4).

FIG. 5. Immunocytochemistry of Cx43 and effects of 4-PB. Immunocytochemistry of human GBM cell cultures hGBM-1 (A), hGBM-5 (B) and hGBM-14 (C) show expression of Cx43. Cells were cultured in 4-PB for 48 hours before fixation and immunostaining, showing Cx43 expression and distribution on each of the 3 cell cultures hGBM-1 (D), hGBM-5 (E) and hGBM-14 (F). Note the marked increase of connexin 43 expression and its redistribution to cytoplasmatic processes shown in (D), (E) and (F).

FIG. 6. Western blot of Cx43 and effects of 4-PB. Human glioblastoma cell cultures hGBM-1, hGBM-5 and hGBM-14 express phosphorylated isoforms of Cx43. Westem blot analysis of Cx43 expression in protein extracts (30 μg/lane) in the presence and absence of 4-PB in human glioblastoma hGBM-1 (A) and hGBM-5 (B) and hGBM-14(C) cells. Anti-Cx43 positive proteins correspond to native non-phosphorylated (P0) and phosporylated (P1-P2) (phosphorylated isoforms grouped together) species of Cx43. Note the marked increase of both Cx43 isoforms in the presence of 4-PB. Statistical significance between means was assessed by Student's t-test for unpaired values. *P<0.05 relative to control untreated cells (n=4).

FIG. 7. Gap iunction-mediated fluorescent dVe transfer in the presence and absence of 4-PB visualized by fluorescence microscopy. Tumour cells (hGBM-1) were preloaded with Dil (red) and calcein (green) fluorescent probes and plated on top of unlabelled cells of the same culture according to Materials and Methods. Labelled cells were allowed to settle. While Dil was retained in the pre-loaded cells, as seen through the red filter (A), green calcein fluorescence, as seen through the green filter, had spread to Dil-negative cells, indicating gap junction-mediated transfer (B). In a parallel experiment, (C-F), cells preloaded with Dil and calcein were plated on top of unlabelled cells, which had been in contact with 4-PB for 48 h. After 6 hours the cells were photographed through the microscope. (C) and (D) show the control experiment without 4-PB similar to (A) and (B) while (E) and (F) show that numerous unlabelled cells had now received calcein. By comparing (D) and (F) it is seen that 4-PB facilitated gap junction dependent calcein transfer substantially. White arrows indicate examples of dye transfer.

FIG. 8. Connexin 43 expression after 4-PB treatment

Protein from S6 cells treated with 4-PB for 48 h was extracted and a Western blot analysis was performed. The intensity of the Cx43 specific signal was determined by imager and the activation of Cx43 was calculated. S6 cells not treated with 4-PB acted as reference.

FIG. 9. The GCV/TK related cell death is time dependent

Mixtures of cell dones were treated with drugs as indicated. After 96 h (grey bar), 120 h (white bar) and 168 h (black bar) MTT tests from three samples were performed. The ratios of the MTT tests from TK negative and TK positve mixtures (A1:RFP/S6:RFP) are shown.

FIG. 10. Fluorescent dye transfer

S6 and RFP cells were plated each in two separate dishes with one dish of each cell type treated either with AGA or DMSO. S6 cells of both dishes were incubated with Calcein-AM and Dil. Cells were trypsinized and 85% RFP cells were mixed with 15% S6 cells and mixtures were co-cultured in two dishes where one contained cells pretreated with AGA (right panel). After 2.5 hours cells were incubated with Hoechst nuclear stain and Calcein-AM dye transfer from S6 cells to RFP cells was monitored.

Left panel (A): w/o AGA, right panel: with AGA treatment (B), upper row: Calcein-AM stain, middle row: Dil stain, lower row: Hoechst nuclear stain. White arrows indicate example of dye transfer.

FIG. 11. Dye transfer from neural stem cells

Human neural stem cells were prelabeled with Dil and Calcein-AM as described in Materials and Methods. Two hours after plating stem cells with glioblastoma cells, transfer of Calcein-AM to non-labelled tumour cells can be seen (indicated by white arrows).

FIG. 12. 4-PB enhanced dve transfer from neural stem cells to glioblastoma cells Human neural stem cells were prelabeled with Dil and Calcein-AM as described in Materials and Methods. A few hours after mixing of stem cells with glioblastoma cells, transfer of Calcein-AM can be seen. White arrows indicate glioblastoma cells labeled with calcein from neural stem cells. A; Untreated control. B; Stem cells and glioblastoma cells pretreated with 4-PB prior to dye transfer analysis. White arrows indicate examples of glioblastoma cells that have received calcein-AM.

FIG. 13. Induction of Connexin 43 in neural stem cells bv 4-PB Neural stem cells were grown to around 50% confluence at which state 0.5 mM 4-PB treatment was started and continued for up to 4 days. Western blot analysis of connexin 43 was performed and the relative amount of protein quantified.

FIG. 14 DLCXSN MMLV derived replication defective VSV-G psudo-typed retrovirus vector and resulting tomato thymidine kinase 1 retrovirus.

For details, see example 3b.

FIG. 15 Tomato thymidine kinase transduction of a glioblastoma cell line enhances toxicity of AZT.

U87MG glioblastoma cell line was transduced with the Tomato thymidine kinase vector of FIG. 14. Transduced (FIG. 15B) and non-transduced (FIG. 15A) cells were exposed to increasing levels of AZT and cell killing was measured. IC50 for non-transduced cells was 7.4286 mM for tomato TK transduced cells 0.0148 mM.

FIG. 16. 4-PB enhances the cell killing effects of AZT in glioma cells expressing a tomato thymidine kinase.

U87 MG wt. cells and U87 MG cells recombinantly expressing the tomato thymidine kinase ZG59, were treated with AZT (15 μM), with 4-phenylbutyrate (0-8 mM) or in combination for 72 hours. Cell killing was assessed by XTT analysis.

### DETAILED DESCRIPTION

Definitions: “A pharmaceutically acceptable prodrug” is a compound that may be converted under physiological conditions or by solvolysis to the specified compound or to a pharmaceutically acceptable salt of such compound.

Pharmaceutically Acceptable Salts

The chemical compounds of the invention may be provided in any form suitable for the intended administration. Suitable forms include pharmaceutically (i.e. physiologically) acceptable salts, and pre- or prodrug forms of the chemical compounds of the invention.

Examples of pharmaceutically acceptable addition salts include, without limitation, the non-toxic inorganic and organic acid addition salts such as the hydrochloride, the hydrobromide, the nitrate, the perchlorate, the phosphate, the sulphate, the formate, the acetate, the aconate, the ascorbate, the benzenesulphonate, the benzoate, the cinnamate, the citrate, the embonate, the enantate, the fumarate, the glutamate, the glycolate, the lactate, the maleate, the malonate, the mandelate, the methanesulphonate, the naphthalene-2-sulphonate derived, the phthalate, the salicylate, the sorbate, the stearate, the succinate, the tartrate, the toluene-p-sulphonate, and the like. Such salts may be formed by procedures well known and described in the art.

Examples of pharmaceutically acceptable cationic salts of a chemical compound of the invention include, without limitation, the sodium, the potassium, the calcium, the magnesium, the zinc, the aluminium, the lithium, the choline, the lysine, and the ammonium salt, and the like, of a chemical compound of the invention containing an anionic group. Such cationic salts may be formed by procedures well known and described in the art.

Sequence Identity

In the context of this invention “identity” is a measure of the degree of identical amino acid residues among sequences. In order to characterize the identity, subject sequences are aligned so that the highest order homology (match) is obtained. Based on these general principles the “percent identity” of two amino acid sequences is determined using the BLASTP algorithm [Tatiana A. Tatusova, Thomas L. Madden: Blast 2 sequences—a new tool for comparing protein and nucleotide sequences; FEMS Microbiol. Lett. 1999 174 247-250], which is available from the National Center for Biotechnology Information (NCBI) web site, and using the default settings suggested here (i.e. Matrix=Blosum62; Open gap=−11; Extension gap=−1; Penalties gap x_dropoff=50; Expect=10; Word size=3; Filter on). The BLAST algorithm determines the % sequence identity in a range of overlap between two aligned sequences. For the purposes of the present invention, the percent sequence identity is preferably calculated in a range of overlap of at least 50 amino acids, more preferably at least 75 amino acids, more preferably at least 100 amino acids, the range being calculated by BLASTP under default settings.

In the context of this invention, “identity” is a measure of the degree of homology of nucleotide sequences. In order to characterize the identity, subject sequences are aligned so that the highest order homology (match) is obtained. Based on these general principles, the “percent identity” of two amino acid sequences or of two nucleic acids is determined using the BLASTN algorithm [Tatiana A. Tatusova, Thomas L. Madden: Blast 2 sequences—a new tool for comparing protein and nudeotide sequences; FEMS Microbiol. Lett. 1999 174 247-250], which is available from the National Center for Biotechnology Information (NCBI) web site, and using the default settings suggested here (i.e. Reward for a match=1; Penalty for a mismatch−−2; Strand option=both strands; Open gap=−5; Extension gap=−2; Penalties gap x_dropoff=50; Expect=10; Word size=11; Filter on). The BLASTN algorithm determines the % sequence identity in a range of overlap between two aligned nucleotide sequences. For the purposes of the present invention the percent sequence identity is preferably calculated in a range of overlap of at least 100 nudeotides, the range being determined by BLASTN under default settings. More preferably the range of overlap is at least 300 nucleotides.

Deoxyribonucleoside Kinase

DNA is made of four deoxyribonucleoside triphosphates, provided by the de novo and the salvage pathway. The key enzyme of the de novo pathway is ribonucleotide reductase, which catalyses the reduction of the 2′-OH group of the nucleoside diphosphates, and the key salvage enzymes are the deoxyribonucleoside kinases, which phosphorylate deoxyribonudeosides to the corresponding deoxyribonucleoside monophosphates. According to the present invention a deoxyribonucleoside kinase is an enzyme capable of catalysing phosophorylation of at least one deoxyribonucleoside or deoxyribonucleoside analogue.

Nucleoside Analogue

A nudeoside analogue is defined as compound comprising a deoxyribonucleoside structure, which compound is substituted in relation to a naturally occurring deoxyribonucleoside either on the deoxyribose part of in the purine or pyrimidine ring. A nucleoside analogue is essentially non-toxic in its non-phosphorylated (nucleoside) state. Analogs of the naturally occurring nucleosides are usually administered as prodrugs, e.g. unphosphorylated, as the omission of the negative charges from the phosphate groups allows effective transport of the analog into the cell. Once prodrugs are converted into a potent cytotoxic metabolite they inhibit or disrupt DNA synthesis. The tumor cells subsequently die via necrotic or apoptotic pathways.

Hybridisation

Hybridization should be accomplished under at least low stringency conditions, but preferably at medium or high stringency conditions.

Suitable experimental conditions for determining hybridisation at low, medium, or high stringency conditions, respectively, between a nucleotide probe and a homologous DNA or RNA sequence, involves pre-soaking of the filter containing the DNA fragments or RNA to hybridise in 5×SSC [Sodium chloride/Sodium citrate; cf. Sambrook et al; Molecular Cloning: A Laboratory Manual, 2^{nd} Ed., Cold Spring Harbor Lab., Cold Spring Harbor, N.Y. 1989] for 10 minutes, and prehybridization of the filter in a solution of 5×SSC, 5× Denhardt's solution [cf. Sambrook et al.; Op cit.], 0.5% SDS and 100 μg/ml of denatured sonicated salmon sperm DNA [cf. Sambrook et al; Op cit.], followed by hybridisation in the same solution containing a concentration of 10 ng/ml of a random-primed [Feinberg A P & Vogelstein B; Anal. Biochem. 1983 132 6-13], ³²P-dCTP-labeled (specific activity>1×10⁹ cpm/μg) probe for 12 hours at approximately 45° C.

The filter is then washed twice for 30 minutes in 2×SSC, 0.5% SDS at a temperature of at least 55° C. (low stringency conditions), more preferred of at least 60° C. (medium stringency conditions), still more preferred of at least 65° C. (medium/high stringency conditions), even more preferred of at least 70° C. (high stringency conditions), and yet more preferred of at least 75° C. (very high stringency conditions).

Molecules to which the oligonudeotide probe hybridises under these conditions may be labelled to detect hybridisation. The complementary nucleic acids or signal nucleic acids may be labelled by conventional methods known in the art to detect the presence of hybridised oligonucleotides. The most common method of detection is the use of autoradiography with e.g. ³H, ¹²⁵I, ³⁵S, ¹⁴C, or ³²P-labelled probes, which may then be detected using an X-ray film. Other labels include ligands, which bind to labelled antibodies, fluorophores, chemoluminescent agents, enzymes, or antibodies, which can then serve as specific binding pair members for a labelled ligand.

Phenylbutyrate, Phenylacetate

Compounds which are capable of enhancing gap-junction communication comprise compounds of the formula: 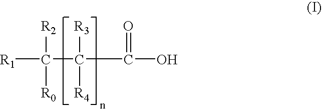
wherein R₀ is aryl (e.g., phenyl, napthyl), phenoxy, substituted aryl (e.g., one or more halogen [e.g., F, Cl, Br, I], lower alkyl [e.g., methyl, ethyl, propyl, butyl] or hydroxy substituents) or substituted phenoxy (e.g., one or more halogen [e.g., F, Cl, Br, I], lower alkyl [e.g., methyl, ethyl, propyl, butyl] or hydroxy substituents);
R₁ and R₂ are each H, lower alkoxy (e.g., methoxy, ethoxy), lower straight and branched chain alkyl (e.g., methyl, ethyl, propyl, butyl) or halogen (e.g., F, Cl, Br, I);
R₃ and R₄ are each H, lower straight and branched chain alkyl (e.g., methyl, ethyl, propyl, butyl), lower alkoxy (e.g., methoxy, ethoxy) or halogen (e.g., F, Cl, Br, I); and
n is an integer from 0 to 2;
salts thereof (e.g., Na⁺, K⁺ or other pharmaceutically acceptable salts); stereoisomers thereof; and mixtures thereof.

When n is equal to 2, each of the two R₃ substituents and each of the two R₄ substituents can vary independently within the above phenylacetic acid derivative definition. It is intended that this definition includes phenylacetic acid (PA) and phenylbutyric acid (PB). Mixtures according to this definition are intended to include mixtures of carboxylic acid salts, for instance, a mixture of sodium phenylacetate and potassium phenylacetate. References herein to a carboxylate, such as phenylacetate (PA) or phenylbutyrate (PB), are intended to refer also to an appropriate counter cation, such as Na⁺, K⁺ or another pharmaceutically acceptable cation such as an organic cation (e.g., arginine). Thus, as used herein, a PA or PB derivative or analog refers to the phenylacetic acid derivatives of this definition. Some of these derivatives can be interconverted when present in a biological system. For instance, PA can be enzymatically converted to PB within an animal and, similarly, PB can be converted to PA. A number of the compounds falling under the generic formula I above have been shown to have equivalent effects to PB and PA in vitro and in vivo (U.S. Pat. No. 5,605,930).

In a more preferred embodiment, the compounds capable of enhancing gap-junction communication refer to a compound of formula I, wherein
R₀=aryl, phenoxy, substituted aryl or substituted phenoxy;
R₁ and R₂=H, lower alkoxy, lower straight and branched chain alkyl or halogen;
R₃ and R₄=H, lower alkoxy, lower straight and branched chain alkyl or halogen; and
n=an integer from 0 to 2;
salts thereof; stereoisomers thereof; and mixtures thereof.

Thus, phenylacetic acid derivatives include, without limitation, phenylacetic acid, phenylpropionic acid, phenylbutyric acid, 1-naphthylacetic acid, phenoxyacetic acid, phenoxypropionic acid, phenoxybutyric acid, 4-chlorophenylacetic acid, 4-chlorophenylbutyric acid, 4-iodophenylacetic acid, 4-iodophenylbutyric acid, α-methylphenylacetic acid, α-methoxyphenylacetic acid, α-ethylphenylacetic acid, α-hydroxyphenylacetic acid, 4-fluorophenylacetic acid, 4-fluorophenylbutyric acid, 2-methylphenylacetic acid, 3-methylphenylacetic acid, 4-methylphenylacetic acid, 3-chlorophenylacetic acid, 3-chlorophenylbutyric acid, 2-chlorophenylacetic acid, 2-chlorophenylbutyric acid and 2,6-dichlorophenylacetic acid, and the sodium and potassium salts of the these compounds.

Among the PA and PB derivatives the most preferred are 4-phenylbutyric acid, phenylacetic acid or their pharmaceutically acceptable salts as these have been the subject of numerous clinical safety trials, and as they are tolerated well by human beings in mM amounts. The most preferred compound is 4-phenylbutyrate or one of its pharmaceutically acceptable salts, because 4-phenylbutyrate is tolerated well and has been tested in numerous clinical trials. Of the salts, sodium and potassium 4-phenylbutyrate is most preferred.

It is also contemplated that the organic acids to be used in the present invention can be administered as the corresponding anhydride, ester or amide pro-drugs, which may be converted into the corresponding salt in vivo. Esters include lower alkyl esters (methyl, ethyl, propyl, butyl), which may be substituted with one or more halogens for increased solubility. In the case of amides, the nitrogen atom may also be substituted. Anhydrides are less preferred due to their inherent instability.

In another embodiment of the invention, the compounds capable of enhancing gap-junction communication comprise valproic acid, a pharmaceutically acceptable salt thereof, an ester, an amide, or anhydrides thereof. Valproic acid and its salts like 4-PB are approved drugs that can be tolerated in large amounts by patients.

It is also contemplated to use longer chain phenyl fatty acids (PA, 4-PB, 6-phenyl-hexanoic, 8-phenyl-octanoic, 10-phenyl-decanoic, etc, as these can be converted through betaoxidation into 4-PB. However the C-6 and longer aromatic fatty acids are less soluble than 4-PB and therefore less preferred.

Further examples of compounds which are believed to enhance gap-junction communication include splitomicin (Bedalov et al, Proc Natl Acad Sci USA. 2001 Dec. 18; 98(26):15113-8), Trichostatin A (1. Merck Index., 13th Edition: 9722, page 1720), and butyric acid and pharmaceutically acceptable salts or prodrugs of any of these. The ability to enhance gap-junction communication can be verified in dye transfer experiments as described in the appended examples or by determining their enhancement of the cytotoxic effect of nucleoside analogues (Example 3 herein).

It is also contemplated that two or more different compounds capable of enhancing gap-junction communication can be administered to the same individual. The composition according to the invention may thus comprise at least three compounds capable of enhancing gap-junction communication, such as at least 3 compounds, for example at least 4 compounds, such as at least 5 compounds.

Deoxyribonucleoside Kinases

Deoxyribonucleoside kinases (dNK) from various organisms differ in their substrate specificity, regulation of gene expression and cellular localisation. In mammalian cells there are four enzymes with overlapping specificities, the thymidine kinases 1 (TK1) and 2 (TK2), deoxycytidine kinase (dCK) and deoxyguanosine kinase (dGK) phosphorylate purine and pyrimidine deoxyribonucleosides. TK1 and TK2 are pyrimidine specific and phosphorylate deoxyuridine (dUrd) and thymidine (dThd), and TK2 also phosphorylates deoxycytidine (dCyd). dCK phosphorylates dCyd, deoxyadenosine (dAdo) and deoxyguanosine (dGuo), but not dThd. dGK phosphorylates dGuo and dAdo. In mammals, TK1 is cytosolic, and TK2 and dGK are localised in the mitochondria, although recent reports indicate a cytoplasmic localisation of TK2 as well.

The best known and most studied example of suicide gene therapy is Herpes simplex virus (HSV) thymidine kinase (tk) gene (Karreman, 1998, A new set of positive/negative selectable markers for mammalian cells. Gene. 218: 57-61). The HSV tk gene leads to cell death when growing cells are exposed to antiherpetic nucleoside analogs such as ganciclovir (GCV), as this and other prodrugs are metabolised by HSV TK to toxic metabolites.

A Drosophila melanogaster deoxyribonucleoside kinase (Dm-dNK) phosphorylates all four natural deoxyribonucleosides as well as several nudeoside analogs (Munch-Petersen et al., 1998, Four deoxynucleoside kinase activities from Drosophila melanogaster are contained within a single monomeric enzyme, a new multifunctional deoxynudeoside kinase. J Biol Chem. 273: 3926-31; Munch-Petersen et al 2000, Functional expression of a multisubstrate deoxyribonudeoside kinase from Drosophila melanogaster and its C-terminal deletion mutants. J Biol Chem. 275: 6673-9; WO 00/36099 “New medical use of gene and vector encoding a multisubstrate deoxyribonucleoside kinase (dNK)”). The broad substrate specificity of this enzyme together with a high catalytic rate makes it unique among the nucleoside kinases for use as a suicide gene in combined gene/chemotherapy of cancer.

Mutant forms of the Drosophila melanogaster Dm dNK have been developed, which have broad substrate specificities (WO 01/88106 “Multi-substrate insect deoxynucleoside kinase variants”). A particularly preferred variant is the variant B5 because its degree of activation is 35 approximately 50 times better than wild type Dm dNK for gemcitabine. The degree of activation is defined as the ratio of the IC₅₀ of the prodrug in the nontransfected cell line to the IC₅₀ of the nudeoside analogue in the transfected cell line.

Examples of deoxyribonucleoside kinases that can be used in the context of the present invention include human TK1 and TK2 and human dCK and human dGK. These and other recombinant kinases in a gene therapy approach can be overexpressed in the tumour cells by placing them under the control of a strong constitutive or tissue specific promoter, such as the CMV promoter, human UbiC promoter, JeT promoter (U.S. Pat. No. 6,555,674), SV40 promoter, and Elongation Factor 1 alpha promoter (EF1-alpha). Another type of preferred promoters include tissue specific promoters, which preferably encompass promoters that are expressed specifically in cancer cells (e.g. the intermediate filament protein nestin promoter promotes cell-specific expression in neuro-epithelial cells of stem cell or malignant phenotype (Lothian, C. et al., 1999, Identification of both general and region-specific embryonic CNS enhancer elements in the nestin promote, Exp. Cell Res., 248:509-519). Other suitable examples of tissue specific promoters indude:
PSA prostate specific antigen (prostate cancer)
AFP Alpha-Fetoprotein (hepatocellular carcinoma)
CEA Carcinoembrionic antigen (epithelial cancers)
COX-2 Cyclooxygenase 2 (tumour)
MUC1 Mucin-like glycoprotein (carcinoma cells)
E2F-1E2F transcription factor 1 (tumour)

Non-limiting examples of specific known sequences of deoxyribonucleoside kinases comprise for example the following:

| HSV-tk wild type ACCESSION V00470 | |
|---|---|
| (SEQ ID NO 1) | |
| MASYPGHQHASAFDQAARSRGHSNRRTALRPRRQQEATEVRPEQKMPTLLRVYIDGPHGMGKTTTTQLLVALGSRD | |
| DIVYVPEPMTYWRVLGASETIANIYTTQHRLDQGEISAGDAAVVMTSAQITMGMPYAVTDAVLAPHIGGEAGSSHA | |
| PPPALTLIFDRHPIAALLCYPAARYLMGSMTPQAVLAFVALIPPTLPGTNIVLGALPEDRHIDRLAKRQRPGERLD | |
| LAMLAAIRRVYGLLANTVRYLQCGGSWREDWGQLSGTAVPPQGAEPQSNAGPRPHIGDTLFTLFRAPELLAPNGDL | |
| YNVFAWALDVLAKRLRSMHVFILDYDQSFAGCRDALLQLTSGMVQTHVTTPGSIPTICDLARTFAREMGEAN | |
| Drosophila melanogaster wildtype kinase GenBanK ACCN Y18048 | |
| (SEQ ID NO 2) | |
| MAEAASCARKGTKYAEGTQPFTVLIEGNIGSGKTTYLNHFEKYKNDICLLTEPVEKWRNVNGVNLLELMYKDPKKW | |
| AMPFQSYVTLTMLQSHTAPTNKKLKIMERSIFSARYCFVENNRRNGSLEQGMYNTLEEWYKFIEESIHVQADLIIY | |
| LRTSPEVAYERIRQRARSEESCVPLKYLQELHELHEDWLIHQRRPQSCKVLVLDADLNLENIGTEYQRSESSIFDA | |
| ISSNQQPSPVLVSPSRRQRVAR | |
| Tomato TK | |
| (SEQ ID NO 3) | |
| MAFSSSARNPVDLRNGSKNSFCPVGEIHVIVGPMFAGKTTALLRRVNLESNDGRNVVLIKSSKDARYAVDAVVTHD | |
| GTRFPCWSLPDLSSFKQRFGKDAYEKVDVIGIDEAQFFGDLYEFCCNAADFDGKIIVVAGLDGDYLRKSFGSVLDI | |
| IPLADTVTKLTARCELCNRRAFFTFRKTNETETELIGGADIYMPVCRQHYVNGQSVNESAKMVLESHKVSNELILE | |
| SPLVDP | |
| Arabidopsis thaliana dNK | |
| (SEQ ID NO 4) | |
| MVDYLRSSVGIIHRNHAESITTFIKESVDDELKDSGPEPNLNVKKRLTFCVEGNISVGKSTFLQRIANETVELQDL | |
| VEIVPEPVDKWQDVGPDHFNILDAFYSEPQRYAYTFQNYVFVTRLMQEKESASGVKPLRLMERSVFSDRMVFVRAV | |
| HEAKWMNEMEISIYDSWFDPVVSSLPGLVFDGFIYLRASPDTCHKRMMLRKRAEEGGVSLKYLQDLHEKHESWLLP | |
| FESGNHGVLSVSRPSLHMDNSLHPDIKDRVFYLEGNHMHSSIQKVPALVLDCEPNIDFSRDIEAKTQYARQVAEFF | |
| EFVKKKQETSTEKSNSQSPVLLPHQNGGLWMGPAGNHVFGLDLPPLDLKSLLTRPSA | |
| Drosophila melanogaster, mutant B5 | |
| (SEQ ID NO 5) | |
| MAEAASCARKGTKYAEGTQPFTVLIEGNIGSGKTTYLNHFEKYKNDICLLTEPVEKWRNVNGVNLLELMYKDPKKW | |
| AMPFQSYATLTMLQSHTAPTNKKLKIMERSIFSARYCFVENMRRNGSLEQGMYNTLEEWYKFIEESIHVQADLIIY | |
| LRTSPEVAYERIRQRARSEESCVPLKYLQELHELHEDWLIHQRRPQSCKVLVLDADLDLENIGTEYQRSESSIFDA | |
| ISSNQQFSPVPVSPSKRQRVAR | |
| >Arabidopsis thaliana dCGK NP_565032 | |
| (SEQ ID NO 6) | |
| 1 | mqkilckstt sstpvlstpv nslaagfisl gfktpvknlp pcsttkplst cffstsampt |
| 61 | ttasvssggv gfsaylqrtv hkpapasvrf stagyrtcrc sidgtnrawv grtgswralf |
| 121 | csdstggltp vnatagavve seeesdgede deekdekpvr mnrrnrsssg sgefvgnpdl |
| 181 | lkipgvqlrn qrklvdngig dvaelkklyk dkfwkasgkm vdylrssvgi ihrnhaesit |
| 241 | tfikesvdde lkdsgpepnl nvkkrltfcv egnisvgkst flqrianetv elqdlveivp |
| 301 | epvdkwqdvg pdhfnildaf ysepqryayt fqnyvfvtrl mqekesasgv kplrlmersv |
| 361 | fsdrmvfvra vheakwmnem eisiydswfd pvvsslpglv pdgfiylras pdtchkrmml |
| 421 | rkraeeggvs lkylqdlhek heswllpfes gnhgvlsvsr pslhmdnslh pdikdrvfyl |
| 481 | egnhmhssiq kvpalvldce pnidfsrdie aktqyarqva effefvkkkq etsteksnsq |
| 541 | spvllphqng glwmgpagnh vpgldlppld lkslltrpsa |
| >Oryza sativa dCGK BAB86213 | |
| (SEQ ID NO 7) | |
| 1 | mveflqssvg iihknhaesi tlfikesvde elkgtdspnv sknkrltfcv egnisvgktt |
| 61 | flgrianeti elrdlveivp epiakwqdvg pdhfnildaf yaepqryayt fqnyvfvtrv |
| 121 | mqekesssgi kplrlmersv fsdrmvvkfl kvfvravhea nwmnemeisi ydswfdpvvs |
| 181 | slpglipdgf iylraspdtc hkrmmvrkrs eeggvtldyl rglhekhesw llpskgqgpg |
| 241 | vlsvsqvpvh megslppdir ervfyleqdh mhssiqkvpa lvldcehdid fnkdieakrq |
| >H. sapiens dCK XP_003471 | |
| (SEQ ID NO 8) | |
| MATPPKRSCPSFSASSEGTRIKKISIEGNIAAGKSTFVNILKQLCEDWEVVPEPVARWCNVQSTQDEFEELTMSQK | |
| NGGNVLQMMYEKPERWSFTFQTYACLSRIRAQLASLNGKLKDAEKPVLFFERSVYSDRYIFASNLYESECMNETEW | |
| TIYQDWHDWMNNQFGQSLELDGIIYLQATPETCLHRIYLRGRNEEQGIPLEYLEKLHYKHESWLLHRTLKTNFDYL | |
| QEVPILTLDVNEDFKDKYESLVEKVKEFLSTL | |
| >H. sapiens dGK XP_002341 | |
| (SEQ ID NO 9) | |
| MAAGRLFLSRLRAPFSSMAKSPLEGVSSSRGLHAGRGPRRLSIEGNIAVGKSTFVKLLTKTYPEWHVATEPVATWQ | |
| NIQAAGNQKACTAQSLGNLLDMMYREPARWSYTFQTFSFLSRLKVQLEPFPEKLLQAPKPVQIFERSVYSDRYIFA | |
| KNLFENGSLSDIEWHIYQDWHSFLLWEFASRITLHGFIYLQASPQVCLKRLYQRAREEEKGIELAYLEQLHGQHEA | |
| WLIHKTTKLHFEALMNIPVLVLDVNDDFSEEVTKQEDLMREVNTFVKNL | |
| >H. sapiens TK2 NP_004605 | |
| (SEQ ID NO 10) | |
| MGAFCQRPSSDKEQEKEKKSVICVEGNIAGGKTTCLEFFSNATDVEVLTEPVSKWRNVRGHNPLGLMYHDASRWGL | |
| TLQTYVQLTMLDRHTRPQVSSVRLMERSIHSARYIFVENLYRSGKMPEVDYVVLSEWFDWILRNMDVSVDLIVYLR | |
| TNPETCYQRLKKRCREEEKVIPLEYLEAIHHLHEEWLIKGSLFPMAAPVLVIEADHHMERMLELFEQNRDRILTPE | |
| NRKHCP | |
| >H. sapiens TK1 XP_037195 | |
| (SEQ ID NO 11) | |
| MSCINLPTVLPGSPSKTRGQIQVILGPMFSGKSTELMRRVRRFQIAQYKCLVIKYAKDTRYSSSFCTHDRNTMEAL | |
| PACLLRDVAQEALGVAVIGIDEGQFFPDIMEFCEAMANAGKTVIVAALDGTFQRKPFGAILNLVPLAESVVKLTAV | |
| CMECFREAAYTKRLGTEKEVEVIGGADKYHSVCRLCYFKKASGQPAGPDNKENCPVPGKFGEAVAARKLFAPQQIL | |
| QCSPAN | |
| >Bombyx mori dNK AAK28318 | |
| (SEQ ID NO 12) | |
| 1 | msannvkpft vfvegnigsg kttflehfrq feditlltep vemwrdlkgc nllelmykdp |
| 61 | ekwamtfqsy vsltmldmhr rpaptpvklm erslfsaryc fvehimrnnt lhpaqfavld |
| 121 | ewfrfiqhni pidadlivyl ktspsivyqr ikkrarseeq cvplsyieel hrlhedwlin |
| 181 | rihaecpapv lvldadldls qitdeykrse hqilrkavnv vmsspnkhsp kkpisttpik |
| 241 | itphmril |

>Anopheles dNK AA049462
(SEQ ID NO 13)
MPPIASEKLGASGKKPFTVFVEGNIGSGKTTFLNHFQKFNDICLLTEPVEKWRNCGGVNLLDLMYKESHRWAMPFQ
TYVTLTMLDMHTCQTDKSVKLMERSLFSARNCFVESMLASGSLHQGMYNVLQEWYDFICCNIHIQADLIVYLQTSP
EVVYERMKQRARSEESCVPLEYLKELHELHENWLIHGASPRPAPVLVLNADLDLNTIGAEYERSETSILKPILIEN
TNQHAILTSPAKRAKTDF
>Rice TK1
(SEQ ID NO 14)
MSSICAMRSLLAASTFLRSGASPLLRPLSRPLPSRLNLSRFGPVRPVSAAAAAADKSRGGGGSAMEAQPSYPGEIH
VIVGPMFAGKTTALLRRVQVEAGTGRNVALIKSDKDNRYGLDSVVTHDGTKMPCWALPELSSFQDKLGTEAYDKVD
VIGIDEAQFFDDLHDFCCKAADRDGKIVVVAGLDGDYKRNKFGSVLDIIPLADSVTKLTARCELCGRRAFFTLRKT
RETKTELIGGADVYMPVCRQHYLDGQIVIEATRIVLDLEKSKVIHAFK
>A. thaliana TK1 AAF13097
(SEQ ID NO 15)
MATLKASFLIKTLDSDVTGDFLSDLERRGSGAVHVIMGPMFSGKSTSLLRRIKSEISDGRSVAMLKSSKDTRYAKD
SVVTHDGIGFPCWALPDLMSFPEKFGLDAYNKLDVIGIDEAQFFGDLYEFCCKVADDDGKIVIVAGLDGDYLRRSF
GAVLDIIPIADSVTKLTARCEVCGHKAFFTLRKNCDTRTELIGGADVYMPVCRKHYITNHIVIKASKKVLEDSDKA
RAESCVAATI
>A. thaliana TK1b
(SEQ ID NO 16)
MRTLISPSLAPFSLHLHKPSLFSTALRFSFSINNITPTNSPPSTISTRKLQTKATRVTSSSSSQPLSSSSPGEIHV
VVGPMFSGKTTTLLRRILAERETGKRIAIIKSNKDTRYCTESIVTHDGEKYPCWSLPDLSSFKERFGFDDYENRLD
VIGIDEAQFFGDLYEFCREAADKEGKTVIVAGLDGDFMRRRFGSVLDLIPIADTVTKLTSRCEVCGKRALFTMRKT
EEKETELIGGAEVYMPVCRSHYVCGQNVLETARAVLDSSNNHSVVASSL
>Tomato dCGK
(SEQ ID NO 17)
MVEFLQSSIGIIHRNHAESITTYIRKSVDEELKENNSDSNVKSTQKKRLTFCVEGNISVGKTTFLQRIANETLELQ
DLVEIVPEPIAKWQDIGPDHFNILDAFYAEPQRYAYTFQNYVFVTRVMQERESSGGIRPLRLMERSVFSDRMVFVR
AVHEANWMNEMEISIYDSWFDPVVSTLPGLIPDGFIYLRASPDTCHKRMMLRKRTEEGGVSLEYLRGLHEKHESWL
FPFESGNHGVLSVSELPLNFDKFCVPPEIRDRVFYLEGNHMHPSIQKVPALVLDCEPNIDFNRDIEAKRQYARQVA
DFFEFVKKKQEVMPGAGEEQPKGNQAPVMLPQNGGLWVPGGKFSESTLNLDFRRNMSFMSH

The corresponding nucleotide sequences can be found in Genbank using the accession numbers given above, in the references given above and for the plant kinases in WO 03/100045 (thymidine kinases), and WO 2004/003185 (dCK/dGK).

In a preferred embodiment, the deoxyribonucleoside kinase is selected from the group consisting of
a) a deoxyribonucleoside kinase having the amino acid sequence of any of SEQ ID No 1 to 17;
b) a deoxyribonucleoside kinase variant comprising an amino acid sequence having at least 50% sequence identity to any of SEQ ID No 1 to 17;
c) a deoxyribonucleoside kinase encoded by a nucleotide sequence capable of hybridising under conditions of high stringency to a nucleotide sequence encoding any of SEQ ID No 1 to 17.

In the context of this invention, the term kinase variant is a polypeptide (or protein) having an amino acid sequence that differs from the sequence presented as SEQ ID NO: 1, as SEQ ID NO:2, as SEQ ID NO: 3, as SEQ ID NO: 4, as SEQ ID NO: 5, as SEQ ID NO: 6, as SEQ ID NO: 7, as SEQ ID NO: 8, as SEQ ID NO: 9, as SEQ ID NO: 10, as SEQ ID NO: 11, as SEQ ID NO: 12, as SEQ ID NO: 13, as SEQ ID NO: 14, as SEQ ID NO: 15, as SEQ ID NO: 16, as SEQ ID NO: 17, at one or more amino acid positions and has dNK activity. Such analogous polypeptides include polypeptides comprising conservative substitutions, splice variants, isoforms, homologues from other species, and polymorphisms.

As defined herein, the term “conservative substitutions” denotes the replacement of an amino acid residue by another, biologically similar residue. Examples of conservative substitutions include
(i) the substitution of one non-polar or hydrophobic residue such as alanine, leucine, isoleucine, valine, proline, methionine, phenylalanine or tryptophan for another, in particular the substitution of alanine, leucine, isoleucine, valine or proline for another; or
(ii) the substitution of one neutral (uncharged) polar residue such as serine, threonine, tyrosine, asparagine, glutamine, or cysteine for another, in particular the substitution of arginine for lysine, glutamic for aspartic acid, or glutamine for asparagine; or
(iii) the substitution of a positively charged residue such as lysine, arginine or histidine for another; or
(iv) the substitution of a negatively charged residue such as aspartic acid or glutamic acid for another.

Modifications of this primary amino acid sequence may result in proteins which have substantially equivalent activity as compared to the unmodified counterpart polypeptide, and thus may be considered functional analogous of the parent proteins. Such modifications may be deliberate, e.g. as by site-directed mutagenesis, or they may occur spontaneous, and include splice variants, isoforms, homologues from other species, and polymorphisms. Such functional analogous are also contemplated according to the invention.

It has been found that deoxyribonucleoside kinase enzymes that are C- and/or N-terminally altered significantly change their properties in particular in respect of kinetic properties such as turnover and substrate specificity. So from having a more restricted specificity, usually deoxycytidine kinase (dCK) and deoxyguanosine kinase (dGK) activity, the deoxyribonucleoside kinase enzymes of the invention may be converted into essentially multi-substrate enzymes, having ability to phosphorylate all four deoxyribonucleosides.

A variant deoxyribonucleoside kinase can be defined with reference to the amino acid sequence of a known deoxyribonucleoside kinase, such as any of the kinases disclosed above. In a preferred embodiment, the variant kinase has at least 50% sequence identity to a reference sequence, more preferably at least 60% sequence identity, more preferably at least 70% sequence identity, more preferably at least 75% sequence identity, more preferably at least 80% sequence identity, more preferably at least 85% sequence identity, more preferably at least 90% sequence identity, more preferably at least 95% sequence identity. The individual reference sequence may be either of SEQ ID NO: 1, SEQ ID NO:2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, and SEQ ID NO: 17.

In a more preferred embodiment, the deoxyribonucleoside kinases comprise a deoxyribonudeoside kinase selected from the group consisting of
a) a deoxyribonucleoside kinase having the amino acid sequence of any of SEQ ID NO 1 to 5; and
b) a deoxyribonucleoside kinase variant comprising an amino acid sequence having at least 70% sequence identity to any of SEQ ID No 1 to 5 and having dNK activity.

It is also possible to administer two or more deoxyribonucleoside kinases to the same individual.

Without being limiting the following combinations of kinase and nucleoside analogues are preferred:
HSV-tk-GCV, ACV, penciclovir
Drosophila melanogaster dNK or B5-gemcitabine, CdA, FaraA, araC, ddC
Plant TKs including Tomato TK-AZT, D4T, ddT, fluorouridine
Plant dNKs including Arabidopsis thaliana dNK-gemcitabine, CdA, FaraA, araC, ddC.
   Method of Treatment

For use in dinical therapy the deoxyribonudeoside kinase enzyme may be administered in any convenient form. In a preferred embodiment, deoxyribonucleoside kinase enzyme is incorporated into a pharmaceutical composition together with one or more adjuvants, excipients, carriers and/or diluents, and the pharmaceutical composition prepared by the skilled person using conventional methods known in the art.

The composition may be administered alone or in combination with one or more other agents, drugs or hormones.

The pharmaceutical composition of this invention may be administered by any suitable route, including, but not limited to oral, intravenous, intramuscular, inter-arterial, intramedullary, intrathecal, intraventricular, transdermal, subcutaneous, intraperitoneal, intranasal, anteral, topical, sublingual or rectal application, buccal, vaginal, intraorbital, intracerebral, intracranial, intraspinal, intraventricular, intracisternal, intracapsular, intrapulmonary, transmucosal, or via inhalation.

Further details on techniques for formulation and administration may be found in the latest edition of Remington's Pharmaceutical Sciences (Maack Publishing Co., Easton, Pa.).

The active ingredient may be administered in one or several doses per day. Currently contemplated appropriate dosages are between 0.5 ng to about 50 μg/kg deoxyribonudeoside kinase/kg body weight per administration, and from about 1.0 ng/kg to about 100 μg/kg daily.

Guidance as to the dosage of gene therapy vector encoding deoxyribonucleoside kinase can be found in the literature concerning clinical and pre-clinical trials in particular with HSV-TK

As for the dosage of the compounds capable of enhancing gap-junction communication, reference can be made to numerous clinical trials of 4-phenylbutyric acid and phenylacetic acid (Carducci et al 2001, Clinical Cancer Research 7:3047-3055; Berg et al, 2001 Cancer Chemother Pharmacol. 2001 May; 47(5):385-90.).

It is currently contemplated that a serum concentration of 0.1 to 100 mM of the compounds capable of enhancing gap-junction communication should be obtained to achieve a therapeutic efficacy. More preferably the serum concentration is 0.5 to 50 mM, more preferably 0.75 to 10 mM, such as from 1-2 mM. The concentration needed to achieve therapeutic efficacy can be determined in vitro. Based on the knowledge concerning the pharmacokinetic properties of phenylbutyrat and phenylacetate, suitable dosage regimes can be predicted and verified in clinical trials. A dosage ranging from 10-1000 mg/kg/day is considered for administration via infusion. More preferably 50-750 mg/kg/day, more preferably 100-500 mg/kg/day. With oral administration 10-100 grams per day for an adult is considered, more preferably 15-50 grams/day, more preferably 20-30 grams/day. These numbers are based on administration of 4-phenylbutyrate (Carducci, M. A. et al., 2001, A Phase I Clinical and Pharmacological Evaluation of Sodium Phenylbutyrate on an 120-h Infusion Schedule Clin. Cancer Res., 7:3047-3055). The dosage ranges of valproic acid are of approximately the same scale as 4-PB or somewhat lower. In case of other gap-junction enhancing compounds, therapeutically effective dosages can be determined in vitro combined with analysis of the pharmacokinetic properties of the compound.

Several nucleoside analogues have been approved by the FDA as drugs and there is ample knowledge concerning the dosages required to obtain therapeutic efficacy for the approved drugs D4T, ddC, AZT, ACV, 3TC, ddA, fludarabine, Cladribine, araC, gemcitabine, Clofarabine, Nelarabine (araG) and Ribarivin. It is considered that the dosage can be reduced compared to known therapeutic regimes by the enhancing effect of co-administration of a compound capable of enhancing gap-junction communication.

The dose administered may be carefully adjusted to the age, weight and condition of the individual being treated, as well as the route of administration, dosage form and regimen, and the result desired, and the practitioner may determine the exact dosage.

In further embodiments, the deoxyribonudeoside kinase may be administered by genetic delivery, using cell lines and vectors as described below under methods of treatment. Vectors can be delivered using liposomes (Yoshida & Mizuno, J Neuro-Oncology, 65:261-267, 2003).

Suitable expression control sequences include promoters, enhancers, transcription terminators, start codons, splicing signals for introns, and stop codons, all maintained in the correct reading frame of the polynucleotide of the invention so as to permit proper translation of mRNA. Expression control sequences may also include additional components such as leader sequences and fusion partner sequences.

The present invention may be used for treating or alleviating a cancer of a living animal body, including a human, which cancer is responsive to the activity of a cytotoxic agent.

The methods and compositions of the present invention may in particular be used as a “suicide gene therapy”. Transfer of a suicide gene to a target cell renders the cell sensitive to compounds or compositions that are relatively non-toxic to normal cells.

The deoxyribonucleoside kinase enzyme may be used directly via e.g., injected, implanted or ingested pharmaceutical compositions to. treat a pathological process responsive to the deoxyribonudeoside kinase enzyme. The naked enzyme may be delivered to the cells using liposome delivery, such as for example the BioPorter® system described in US 20030008813 and US 20030054007. The liposomes can be targeted to cancer cells using ligands for cancer cell surface markers.

Suitable expression vectors may be a viral vector derived from Herpes simplex, adenovira, lentivira, retrovira, or vaccinia vira, or from various bacterially produced plasmids, and may be used for in vivo delivery of nucleotide sequences to a whole organism or a target organ, tissue or cell population. Other methods include, but are not limited to, liposome transfection, electroporation, transfection with carrier peptides containing nuclear or other localising signals, and gene delivery via slow-release systems.

Other suitable expression vectors include general purpose mammalian vectors which are also obtained from commercial sources (Invitrogen Inc., Clonetech, Promega, BD Biosecences, etc) and contain selection for Geneticin/neomycin (G418), hygromycin B, puromycin, Zeocin/bleomycin, blasticidin Si, mycophenolic acid or histidinol.

The vectors include the following classes of vectors: general eukaryotic expression vectors, vectors for stable and transient expression and epitag vectors as well as their TOPO derivatives for fast cloning of desired inserts (see list below for available vectors).
Ecdysone-lnducible Expression:
   pIND(SP1) Vector
   pIND/V5-His Tag Vector Set
   pIND(SP1)V5-His Tag Vector Set
   EcR Cell Lines
   Muristerone A
Stable Expression:
   pcDNA3.1/Hygro
   pSecTag A, B & C
   pcDNA3.1(−)/MycHis A, B & C
   pcDNA3.1 +/−
   pcDNA3.1/Zeo (+) and pcDNA3.1/Zeo (−)
   pcDNA3.1/His A, B, & C
   pRc/CMV2
   pZeoSV2 (+) and pZeoSV2 (−)
   pRc/RSV
   pTracer™-CMV
   pTracer™-SV40
Transient Expression:
   pCDM8
   pcDNA1.1
   pcDNA1.1/Amp
Epitag Vectors:
pcDNA3.1/MycHis A, B & C
pcDNA3.1N5-His A, B, & C

In a preferred embodiment the polynucleotide sequence or the expression vector is administered in vivo.

In a preferred embodiment, the cancer type is multicellular, solid tumor which is more amenable to the enhanced gap-junction communication demonstrated in the present invention.

HSV-tk has been used for treating the following types of cancer, which are particularly amenable to gap-junction enhanced kinase therapy according to the present invention. Bladder cancer, Sutton et al 1997, Urology, 49:173-180; Neuroblastoma, Bi, X and Zhang, J-Z. Pediadtr. Surg. Int., 19:400-405, 2003; Glioblastoma, Germano I. M et al. J. Neurooncol., 65:279-289, 2003; Esophageal cancer, Matsubara, H. and Ochiai, Nippon Rinsho. 2000 September; 58(9):193-543.; Tongue cancer, Wang, J. H. et al. Chin J. Dent. Res. 2000, December. 3(4): 44-48; Hepatocellular carcinoma, Gerolami, R. et al. J. Hepatol. 291-297, 2004; Lung cancer, Kurdow, R. et al. Ann. Thorac. Surg. 2002 March; 73(3):905-910; Malignant melanoma, Yamamoto, S. et al. Cancer Gene Therapy, 10:179-186, 2003; Ovarian cancer, Barnes, M. N. and Pustilnik, T. B. Curr. Opin. Obstet Gynecol., 13:47-51, 2001; Prostate cancer. Kubo, H. et al. Human Gene Therapy., 14:227-241, 2003; Renal cell carcinoma, Pulkkanen, K. J. Cancer Gene Therapy, 9:908-916, 2002.

Preferably, the Cancer is Breast Cancer and Malignant Glioma

Migrating cells that are capable of tracking down glioma cells and that have been engineered to deliver a therapeutic molecule represent an ideal solution to the problem of glioma cells invading normal brain tissue. It has been demonstrated that the migratory capacity of neural stem cells (NSCs) is ideally suited to therapy in neurodegenerative disease models that require brain-wide cell replacement and gene expression. It was hypothesized that NSCs may specifically home to sites of disease within the brain. Studies have also yielded the intriguing observation that transplanted NSCs are able to home into a primary tumor mass when injected at a distance from the tumor itself; furthermore, NSCs were observed to distribute themselves throughout the tumor bed, even migrating in juxtaposition to advancing single tumor cells (Dunn & Black, Neurosurgery 2003, 52:1411-1424; Aboody et al, PNAS, 2000, 97:12846-12851). These authors showed that NSCs were capable of tracking infiltrating glioma cells in the brain tissue peripheral to the tumor mass, and “piggy back” single tumor cells to make cell-to-cell-contact. In addition to stem cells, progenitor cells may also be capable of tracking down cancer cells in vivo.

Engineered NSCs expressing an enzyme that can activate a prodrug can be used to track and destroy advancing glioma cells. This approach would be substantially enhanced if gap junction communication can be increased and thus the bystander killing effect facilitated. We have shown that the short chain aromatic fatty acid 4-PB can induce such gap junction communication between NSCs and glioma cells in vitro (FIG. 12).

Preferably the kind of stem cell used for this type of therapy originates from the same tissue as the tumour cell or from the same growth layer. Stem cells may comprise embryonal stem cells, or more committed stem and/or progenitor cells, such as neural stem cells, precursor cells, and progenitor cells, Alternatively, the stem cells may originate from bone marrow. The stem cells may be isolated from the patient (e.g. bone marrow stem cells), be engineered to over-express a deoxyribonucleoside kinase and be used in the same patient (autograft). For use in the CNS, where graft-host incompatibility does not constitute a significant problem, the cells may originate from a donor (allograft). The donor approach is preferred for the CNS as this makes it possible to produce large quantities of well-characterised stem and/or precursor and/or progenitor cells, which can be stored and are ready for use. It is also contemplated to use xenografts, i.e. stem/progenitor/precursor cells originating from another species, such as other primates or pigs. Cells for xenotransplantation may be engineered to reduce the risk of tissue rejection.

To generate such therapeutic cell lines, the polynucleotide coding for a dNK may be inserted into an expression vector, e.g. a plasmid, virus or other expression vehicle, and operatively linked to expression control sequences by ligation in a way that expression of the coding sequence is achieved under conditions compatible with the expression control sequences.

Encapsulated Gene Therapy

Encapsulated gene therapy is also contemplated by implanting virus-producing cells in a capsule with a jacket as described in U.S. Pat. No. 6,027,721 (Cytotherapeutics) in close proximity to the cancer cells. The viruses are preferably retroviruses as retroviruses transduce only dividing cells and therefore specifically target cancer cells. These capsules allow for the efficient adjustment or termination of gene therapy regimes. Specifically, the jackets of the capsules of this invention comprise membranes that permit passage of viral particles, thereby permitting infection of target host tissue.

The capsules can be manufactured using the methods and materials disclosed in WO 92/19195. Briefly, the capsule is comprised of (a) a core containing isolated packaging cells, either suspended in a liquid medium or immobilized within a biocompatible matrix, and (b) a. biocompatible surrounding or peripheral region (“jacket”) of a material that permits passage of the viral particles. The capsule can be any configuration appropriate for maintaining biological activity of the packaging cells and providing access for delivery of the viral particles, including, for example, cylindrical, rectangular, disk-shaped, patch-shaped, ovoid, stellate, or spherical. Moreover, the capsule can be coiled or wrapped into a mesh-like or nested structure. Certain shapes, such as rectangles, patches, disks, cylinders and flat sheets offer greater structural integrity and are preferable where retrieval is desired.

Although microcapsular devices are contemplated (see, e.g., U.S. Pat. No. 4,353,888, incorporated herein by reference), we prefer that the device be of a sufficient size and durability for complete retrieval after implantation. Such macrocapsules have a core of a preferable minimum volume of about 1 to 10 μL and depending upon use are easily fabricated to have a volume in excess of 100 μL. In a hollow-fiber configuration, the fiber will have an inside diameter of less than 1500 microns, preferably about 300-600 microns.

For ease of retrieval, the capsules preferably have one or more tethers to allow location and grasping of the device without damage to it. Additionally, the tether can be used to find the implanted macrocapsule when it is desirous to terminate therapy, by inclusion of suitable identification means.

Another type of encapsulated gene therapy is described in WO 97/01357 (Bavarian Nordic Research Institute). Briefly, such encapsulated cells producing viral particles allow the release of the viral particles produced by the cells from the capsules, and at the same time do not elicit a significant host immune or inflammatory response after implantation in a host.

The encapsulated cells can be prepared by suspending the cells producing viral particles in an aqueous solution of a polyelectrolyte (e.g. selected from sulphate group-containing polysaccharides or polysaccharide derivatives or of sulphonate group containing synthetic polymers), whereafter the suspension in the form of preformed particles is introduced into a precipitation bath containing an aqueous solution of a counter-charged polyelectrolyte (such as for example a polymer with quaternary ammonium groups).

Such capsules are prepared as described in WO 97/01357. The capsules have a variable diameter between 0.01 and 5 mm, but are preferably between 0.1 and 1 mm. Consequently, capsules can be made to contain a variable number of cells. Using the encapsulation process according to the invention, up to 10¹⁰, but preferably 10⁵-10⁷ cells producing viral partides can be encapsulated in the polyelectrolyte complex. The pore size of the capsules is between 80 and 150 nm, preferably between 100 and 120 nm.

After a suitable period in culture (normally not less than 1 hour and not exceeding 30 days), the cell containing capsules can be surgically implanted either directly, or by injection using a syringe into various areas of the body.

Packaging cells may also be immobilised on a support and implanted close to the tumour using the technology described in U.S. Pat. No. 6,565,845. Briefly, the virus particle producer cells may first be attached in vitro on a support matrix. Materials of which the support matrix can be comprised include those materials to which cells adhere following in vitro incubation, and on which cells can grow, and which can be implanted into a mammal without producing a toxic reaction, or an inflammatory reaction which would destroy the implanted cells or otherwise interfere with their biological or therapeutic activity. Such materials may be synthetic or natural chemical substances or substances having a biological origin. The matrix materials include, but are not limited to, glass and other silicon oxides, polystyrene, polypropylene, polyethylene, polyvinylidene fluoride, polyurethane, polyalginate, polysulphone, polyvinyl alcohol, acrylonitrile polymers, polyacrylamide, polycarbonate, polypentent, nylon, amyloses, gelatin, collagen, natural and modified polysaccharides, including dextrans and celluloses (e.g. nitrocellulose), agar, and magnetite. Either resorbable or non-resorbable materials may be used. Also intended are extracellular matrix materials, which are well-known in the art. Extracellular matrix materials may be obtained commercially or prepared by growing cells which secrete such a matrix, removing the secreting cells, and allowing the cells which are to be transplanted to interact with and adhere to the matrix.

To improve cell adhesion, survival and function, the solid matrix may optionally be coated on its external surface with factors known in the art to promote cell adhesion, growth or survival. Such factors include cell adhesion molecules, extracellular matrix, such as, for example, fibronectin, laminin, collagen, elastin, glycosaminoglycans, or proteoglycans or growth factors, such as, for example, nerve growth factor (NGF).

A preferred form of support matrix is a glass bead. Another preferred bead is a polystyrene bead. Bead sizes may range from about 10 μm to 1 cm in diameter, preferably from about 90 to about 150 μm. For a description of various microcarrier beads, see, for example, Fisher Biotech Source 87-88, Fisher Scientific Co., 1987, pp. 72-75; Sigma Cell Culture Catalog, Sigma Chemical Co., St. Louis, 1991, pp. 162-163; Ventrex Product Catalog, Ventrex Laboratories, 1989. The upper limit on the bead size is dictated by the bead's stimulation of undesired host reactions such as gliosis, which may interfere with the function of the transplanted cells or cause damage to the surrounding tissue.

Prodrugs/Nucleoside Analogs

The present invention. is directed to pharmaceutical compositions and uses of such compositions comprising nucleoside analogs and related compounds, including their prodrugs.

On a functional level, a nucleoside analogue is a compound with a molecular weight less than 1000 Daltons, which is substantially non-toxic to human cells, which can be phosphorylated by a deoxyribonudeoside kinase to mono, di, and tri phosphate, the triphosphate of which is toxic to dividing human cells.

The composition according to the invention may comprise at least two or more different nudeoside analogues, such as at least 3 nucleoside analogues, for example at least 4 nudeoside analogues, such as at least 5 nucleoside analogues.

Numerous nucleoside analogs exist that can be converted into a toxic product including a large group described in US 20040002596.

In a preferred embodiment the nucleoside analogue include a compound selected from the group consisting of aciclovir (9-[2-hydroxy-ethoxy]-methyl-guanosine), buciclovir, famciclovir, ganciclovir (9-[2-hydroxy-1-(hydroxymethyl)ethoxyl-methyl]-guanosine), penciclovir, valciclovir, trifluorothymidine, AZT (3′-azido-3′-thymidine), AIU (5′-iodo-5′-amino-2′,5′-dideoxyuridine), ara-A (adenosine-arabinoside; Vivarabine), ara-C (cytidine-arabinoside), ara-G (9-beta-D-arabinofuranosylguanine), ara-T, 1-beta-D-arabinofuranosyl thymine, 5-ethyl-2′-deoxyuridine, 5-iodo-5′-amino-2,5′-dideoxyuridine, 1-[2-deoxy-2-fluoro-beta-D-arabino furanosyl]-5-iodouracil, idoxuridine (5-iodo-2′deoxyuridine), fludarabine (2-Fluoroadenine 9-beta-D-Arabinofuranoside), gencitabine, 3′-deoxyadenosine (3-dA), 2′,3′-dideoxylnosine (ddI), 2′,3′-dideoxycytidine (ddC), 2′,3′-dideoxythymidine (ddT), 2′,3′-dideoxyadenosine (ddA), 2′,3′-dideoxyguanosine (ddG), 2-chloro-2′-deoxyadenosine (2CdA), 5-fluorodeoxyuridine, BVaraU ((E)-5-(2-bromovinyl)-1-beta-D-arabinofuranosyluracil), BVDU (5-bromovinyl-deoxyuridine), FIAU (1-(2-deoxy-2-fluoro-beta-D-arabinofuranosyl)-5-iodouracil), 3TC (2′-deoxy-3′-thiacytidine), dFdC gemcitabine (2′,2′-difluorodeoxycytidine), dFdG (2′,2′-difluorodeoxyguanosine), 5-fluorodeoxyuridine (FdUrd), d4T (2′,3′didehydro-3′-deoxythymidine), ara-M (6-methoxy purinearabinonucleoside), IudR (5-Jodo-2′deoxyuridine), CaFdA (2-chloro-2-ara-fluoro-deoxyadenosine), ara-U (1-beta-D-arabinofuranosyluracil), FBVAU (E)-5-(2-bromovinyl)-1-(2-deoxy-2-fluoro-beta-D-arabinofuranosyl)uracil, FMAU 1-(2-deoxzy-2-fluoro-beta-D-arabinofuranosyl)-5-methyluracil, FLT 3′-fluoro-2′-deoxythymidine, 5-Br-dUrd 5-bromodeoxyuridine, 5-Cl-dUrd 5-chlorodeoxyuridine, dFdU 2′,2-difluorodeoxyuridine, (−)Carbovir (C-D4G), 2,6-Diamino-ddP (ddDAPR; DAPDDR; 2,6-Diamino-2′,3′-dideoxypurine-9-ribofuranoside), 9-(2′-Azido-2′,3′-dideoxy-β-D-erythropentofuranosyl)adenine (2′-Azido-2′,3′-dideoxyadenosine; 2′-N3ddA), 2′FddT (2′-Fluoro-2′,3′-dideoxy-β-D-erythro-pentofuranosyl)thymine), 2′-N3ddA(β-D-threo) (9-(2′-Azido-2′,3′-dideoxy-β-D-threopentofuranosyl)adenine), 3-(3-Oxo-1-propenyl)AZT (3-(3-Oxo-1-propenyl)-3′-azido-3′-deoxythymidine), 3′-Az-5-Cl-ddC (3′-Azido-2′,3′-dideoxy-5-chlorocytidine), 3′-N3-3′-dT (3′-Azido-3′-deoxy-6-azathymidine), 3′-F-4-Thio-ddT (2′,3′-Dideoxy-3′-fluoro-4-thiothymidine), 3′-F-5-Cl-ddC (2′,3′-Dideoxy-3′-fluoro-5-chlorocytidine), 3′-FddA (B-D-Erythro) (9-(3′-Fluoro-2′,3′-dideoxy-B-D-erythropentafuranosyl)adenine), Uravidine (3′-Azido-2′,3′-dideoxyuridine; AzdU), 3′-FddC (3′-Fluoro-2′,3′-dideoxycytidine), 3′-F-ddDAPR (2,6-Diaminopurine-3′-fluoro-2′,3′-dideoxyriboside), 3′-FddG (3′-Fluoro-2′,3′-dideoxyguanosine), 3′-FddU (3′-Fluoro-2′,3′-dideoxyuridine), 3′-Hydroxymethyl-ddC (2′,3′-Dideoxy-3′-hydroxymethyl cytidine; BEA-005), 3′-N3-5-CF3-ddU (3′-Azido-2′,3′-dideoxy-5-trifluoromethyluridine), 3′-N3-5-Cyanomethyloxy-ddU (3′-Azido-2′,3′-dideoxy-5-[(cyanomethyl)oxy]uridine), 3′-N3-5-F-ddC (3′-Azido-2′,3′-dideoxy-5-fluorocytidine), 3′-N3-5-Me-ddC (CS-92; 3′-Azido-2′,3′-dideoxy-5-methylcytidine), 3′-N3-5-NH2-ddU (3′-Azido-2′,3′-dideoxy-5-aminouridine), 3′-N3-5-NHMe-ddU (3′-Azido-2′,3′-dideoxy-5-methyaminouridine), 3′-N3-5-NMe2-ddU (3′-Azido-2′,3′-dideoxy-5-dimethylaminouridine), 3′-N3-5-OH-ddU (3′-Azido-2′,3′-dideoxy-5-hydroxyuridine), 3′-N3-5-SCN-ddU (3′-Azido-2′,3′-dideoxy-5-thiocyanatouridine), 3′-N3-ddA (9-(3′-Azido-2′,3′-dideoxy-B-D-erythropentafuranosyl)adenine), 3′-N3-ddC (CS-91; 3′-Azido-2′,3′-dideoxycytidine), 3′-N3ddG (AZG; 3′-Azido-2′,3′-dideoxyguanosine), 3′-N3-N4-5-diMe-ddC (3′-Azido-2′,3′-dideoxy-N4-5-dimethylcytidine), 3′-N3-N4-OH-5-Me-ddC (3′-Azido-2′,3′-dideoxy-N4-OH-5-methylcytidine), 4′-Az-3′-dT (4′-Azido-3′-deoxythymidine), 4′-Az-5CldU (4′-Azido-5-chloro-2′-deoxyuridine), 4′-AzdA (4′-Azido-2′-deoxyadenosine), 4′-AzdC (4′-Azido-2′-deoxycytidine), 4′-AzdG (4′-Azido-2′-deoxyguanosine), 4′-AzdI (4′-Azido-2′-deoxylnosine), 4′-AzdU (4′-Azido-2′-deoxyuridine), 4′-Azidothymidine (4′-Azido-2′-deoxy-.beta.-D-erythro-pentofuranosyl-5-methyl-2,4-dioxopyrimidine), 4′-CN-T (4′-Cyanothymidine), 5-Et-ddC (2′,3′-Dideoxy-5-ethylcytidine), 5-F-ddC (5-Fluoro-2′,3′-dideoxycytidine), 6Cl-ddP (D2ClP; 6-Chloro-ddP; CPDDR; 6-Chloro-9-(2,3-dideoxy-.beta.-D-glyceropentofuranosyl)-9H-purine), 935U83 (2′,3′-Dideoxy-3′-fluoro-5-chlorouridine; 5-Chloro-2′,3′-dideoxy-3′-fluorouridine; FddClU; Raluridine), AZddBrU (3′-N3-5-Br-ddU; 3′-Azido-2′,3′-dideoxy-5-bromouridine), AzddClU; AzddClUrd (3′-Azido-5-chloro-2′,3′-dideoxyuridine), AZddEtU (3′-N3-5-EtddU; CS-85; 3′-Azido-2′,3′-dideoxy-5-ethyluridine), AZddFU (3′-Azido-2′,3′-dideoxy-5-fluorouridine), AZddIU (3′-N3-5-I-ddU; 3′-Azido-2′,3′-dideoxy-5-iodouridine), AZT-2,5′-anhydro (2,5′-Anhydro-3′-azido-3′-deoxythymidine), AZT-α-L (α-L-AZT), AZU-2,5′-anhydro (2,5′-Anhydro-3′-azido-2′,3′-dideoxyuridine), C-analog of 3′-N3-ddU (3′-Azido-2′,3′-dideoxy-5-aza-6-deazauridine), D2SMeP (9-(2,3-Dideoxy-β-D-ribofuranosyl)-6-(methylthio)purine), D4A (2′,3′-Dideoxydidehydroadenosine), D4C (2′,3′-Didehydro-3′-deoxycytidine), D4DAP (2,6-Diaminopurine-2′,3′-dideoxydidehydroriboside; ddeDAPR), D4FC (D-D4FC; 2′,3′-Didehydro-2′,3′-dideoxy-5-fluorocytidine), D4G (2′,3′-Didehydro-2′,3′-dideoxyguanosine), DMAPDDR (N-6-dimethyl ddA; 6-Dimethylaminopurine-2′,3′-dideoxyriboside), dOTC (−) ((−)-2′-Deoxy-3′-oxa-4′-thiocytidine), dOTC (+) ((+)-2′-Deoxy-3′-oxa-4′-thiocytidine), dOTFC (−) ((−)-2′-Deoxy-3′-oxa-4′-thio-5-fluorocytidine), dOTFC (+) ((+)-2′-Deoxy-3′-oxa-4′-thio-5-fluorocytidine), DXG ((−)-β-Dioxolane-G), DXC-α-L-(α-L-Dioxalane-C), FddBrU (2′,3′-Dideoxy-3′-fluoro-5-bromouridine), FddlU (3′-Fluoro-2′,3′-dideoxy-5-iodouridine), FddT (Alovudine; 3′-FddT; FddThD; 3′-FLT; FLT), FTC (Emtricitabine; Coviracil; (−)-FTC; (−)-2′,3′-Dideoxy-5-fluoro-3′-thiacytidine), FTC-α-L-(α-L-FTC), L-D4A (L-2′,3′-Didehydro-2′,3′-dideoxyadenosine), L-D4FC (L-2′,3′-Didehydro-2′,3′-dideoxy-5-fluorocytidine), L-D4I (L-2′,3′-Didehydro-2′,3′-dideoxylnosine), L-D4G (L-2′,3′-Didehydro-2′,3′-deoxyguanosine), L-FddC (β-L-5F-ddC), Lodenosine (F-ddA; 2′-FddA (B-D-threo); 2′-F-dd-ara-A; 9-(2′-Fluoro-2′,3′-dideoxy-B-D-threopentafuranosyl)adenine), MeAZddIsoC (5-Methyl-3′-azido-2′,3′-dideoxylsocytidine), N6-Et-ddA (N-Ethyl-2′,3′-dideoxyadenosine), N-6-methyl ddA (N6-Methyl-2′,3′-dideoxyadenosine) or RO31-6840 (1-(2′,3′-Dideoxy-2′-fluoro-β-D-threo-pentofuranosyl)cytosine).

Preferred examples of cytidine, guanosine and adenosine analogs include dFdC gemcitabine (2′,2′-difluorodeoxycytidine), 2-chloro-2′-deoxyadenosine (2CdA), CaFdA (2-chloro-2-ara-fluoro-deoxyadenosine), fludarabine (2-Fluoroadenine 9-beta-D-Arabinofuranoside), 2′,3′-dideoxycytidine (ddC), 2′,3′-dideoxyadenosine (ddA), 2′,3′-dideoxyguanosine (ddG), ara-A (adenosine-arabinoside; Vivarabine), ara-C (cytidine-arabinoside), ara-G (9-beta-D-arabinofuranosylguanine), aciclovir (9-[2-hydroxy-ethoxy]-methyl-guanosine), bucidovir, famciclovir, ganciclovir (9-[2-hydroxy-1-(hydroxymethyl)ethoxyl-methyl]-guanosine), penciclovir, valciclovir, 3TC (2′-deoxy-3′-thiacytidine), dFdG (2′,2′-difluorodeoxyguanosine), 2,6-Diamino-ddP (ddDAPR; DAPDDR; 2,6-Diamino-2′,3′-dideoxypurine-9-riboftiranoside), 9-(2′-Azido-2′,3′-dideoxy-β-D-erythropentofuranosyl)adenine (2′-Azido-2′,3′-dideoxyadenosine; 2′-N3ddA), 2′-N3ddA(β-D-threo) (9-(2′-Azido-2′,3′-dideoxy-β-D-threopentofuranosyl)adenine), 3′-Az-5-Cl-ddC (3′-Azido-2′,3′-dideoxy-5-chlorocytidine), 3′-F-5-Cl-ddC (2′,3′-Dideoxy-3′-fluoro-5-chlorocytidine), 3′-FddA (B-D-Erythro) (9-(3′-Fluoro-2′,3′-dideoxy-B-D-erythropentafuranosyl)adenine), 3′-FddC (3′-Fluoro-2′,3′-dideoxycytidine), 3′-F-ddDAPR (2,6-Diaminopurine-3′-fluoro-2′,3′-dideoxyriboside), 3′-FddG (3′-Fluoro-2′,3′-dideoxyguanosine), 3′-Hydroxymethyl-ddC (2′,3′-Dideoxy-3′-hydroxymethyl cytidine; BEA-005), 3′-N3-5-F-ddC (3′-Azido-2′,3′-dideoxy-5-fluorocytidine), 3′-N3-5-Me-ddC (CS-92; 3′-Azido-2′,3′-dideoxy-5-methylcytidine), 3′-N3-ddA (9-(3′-Azido-2′,3′-dideoxy-B-D-erythropentafuranosyl)adenine), 3′-N3-ddC (CS-91; 3′-Azido-2′,3′-dideoxycytidine), 3′-N3ddG (AZG; 3′-Azido-2′,3′-dideoxyguanosine), 3′-N3-N4-5-diMe-ddC (3′-Azido-2′,3′-dideoxy-N4-5-dimethylcytidine), 3′-N3-N4-OH-5-Me-ddC (3′-Azido-2′,3′-dideoxy-N4-OH-5-methylcytidine), 4′-AzdA (4′-Azido-2′-deoxyadenosine), 4′-AzdC (4′-Azido-2′-deoxycytidine), 4′-AzdG (4′-Azido-2′-deoxyguanosine), 5-Et-ddC (2′,3′-Dideoxy-5-ethylcytidine), 5-F-ddC (5-Fluoro-2′,3′-dideoxycytidine), 6Cl-ddP (D2ClP; 6-Chloro-ddP; CPDDR; 6-Chloro-9-(2,3-dideoxy-.beta.-D-glyceropentofuranosyl)-9H-purine), D2SMeP (9-(2,3-Dideoxy-β-D-ribofuranosyl)-6-(methylthio)purine), D4A (2′,3′-Dideoxydidehydroadenosine), D4C (2′,3′-Didehydro-3′-deoxycytidine), D4DAP (2,6-Diaminopurine-2′,3′-dideoxydidehydroriboside; ddeDAPR), D4FC (D-D4FC; 2′,3′-Didehydro-2′,3′-dideoxy-5-fluorocytidine), D4G (2′,3′-Didehydro-2′,3′-dideoxyguanosine), DMAPDDR (N-6-dimethyl ddA; 6-Dimethylaminopurine-2′,3′-dideoxyriboside), dOTC (−) ((−)-2′-Deoxy-3′-oxa-4′-thiocytidine), dOTC (+) ((+)-2′-Deoxy-3′-oxa-4′-thiocytidine), dOTFC (−) ((−)-2′-Deoxy-3′-oxa-4′-thio-5-fluorocytidine), dOTFC (+) ((+)-2′-Deoxy-3′-oxa-4′-thio-5-fluorocytidine), DXG ((−)-β-Dioxolane-G), DXC-α-L-(α-L-Dioxalane-C), FTC (Emtricitabine; Coviracil; (−)-FTC; (−)-2′,3′-Dideoxy-5-fluoro-3′-thiacytidine), FTC-α-L-(α-L-FTC), L-D4A (L-2′,3′-Didehydro-2′,3′-dideoxyadenosine), L-D4FC (L-2′,3′-Didehydro-2′,3′-dideoxy-5-fluorocytidine), L-D4I (L-2′,3′-Didehydro-2′,3′-dideoxylnosine), L-D4G (L-2′,3′-Didehydro-2′,3′-deoxyguanosine), L-FddC (β-L-5F-ddC), Lodenosine (F-ddA; 2′-FddA (B-D-threo); 2′-F-dd-ara-A; 9-(2′-Fluoro-2′,3′-dideoxy-B-D-threopentafuranosyl)adenine), MeAZddIsoC (5-Methyl-3′-azido-2′,3′-dideoxylsocytidine), N6-Et-ddA (N-Ethyl-2′,3′-dideoxyadenosine), N-6-methyl ddA (N6-Methyl-2′,3′-dideoxyadenosine) or RO31-6840 (1-(2′,3′-Dideoxy-2′-fluoro-β-D-threo-pentofuranosyl)cytosine).

Even more preferred ae D4T, ddC, AZT, ACV, 3TC, ddA Fludarabine, Cladribine, araC, gemcitabine, Clofarabine, Nelarabine (araG), and Ribavirin. These drugs have already been approved by the FDA for cancer treatment.

Even more preferred are gemcitabine and AZT.

### EXAMPLES

### Example 1

### 4-Phenylbutyrate Modulates GFAP and Connexin 43 Expression and Enhances Gap Junction Communication in Human Glioblastoma Cells

Materials and Methods:

Tissue source, processing, and primary culture

Tumour tissue specimens were obtained from patients during open surgical resection of glioblastoma multiforme (GBM, WHO grade IV) according to per-operative diagnosis on cryostat sections. Recovery of tissue was done with the permission of the Ethics Committee of Karolinska Institute and with the consent of the patient. The three GBM cultures, hGBM-1, hGBM-5 and hGBM-14, used in this study, were isolated and characterized as previously described (Asklund et al, Exp cell Res, 2003, 284:183-193). Glioblastoma cells were cultured in DMEM/F12 culture medium supplemented with 10% FCS (Gibco), penicillin/streptomycin (100 U/ml, Gibco).

Assessment of Cell Viability and Cell Proliferation by MTT Assay

Proliferation of cultured glioma cells was assessed by 3-(4,5-dimethylthiazol-2-yl)-2,5-diphenyltetrazolium bromide (MTT, Sigma) assay. MTT tetrazolium salt was dissolved in serum-free culture medium (0.5 mg/ml), added to the cells (150 μl/well) and incubated for 30 min at 37° C. Formazan dye, formed by viable cells, was solubilized in 300 μl of isopropanol. Aliquots (100 μl) of the solutions were transferred to 96-well microplates. The absorbance at 570 nm (with reference at 650 nm) was measured using a microtiter plate spectrophotometer (Anthos HT III). The results were expressed as percentage of viablecells compared to the control sample of untreated cells (100%).

Immunocytochemistry

Tumour cells cultured on laminin/poly-ornithine coated glass chamber slides were washed in phosphate buffered saline (PBS) and fixed in 4% paraformaldehyde in phosphate buffer for 10 minutes. Cells were washed in PBS three times and incubated with primary antibodies diluted in buffer (0.3% TX-100 (Sigma), albumin fraction V (Sigma), phosphate buffer pH 7.0) over night at 4° C. Secondary antibodies conjugated with FITC or Cy3 (Jackson lmmunoresearch Laboratories) were applied for one hour at room temperature, followed by rinsing with PBS. Antibodies bound were visualized with epifluorescence microscopy using a Leica DMRB microscope, and photographs were taken using a Nikon F50 camera.

The following antibodies were used at specified dilutions: monoclonal anti GFAP, 1:500 (DAKO), monodonal anti Cx43 (1:1000, Transduction Laboratories), and FITC (1:80)-conjugated secondary antibodies (Jackson lmmunoresearch Laboratories).

Western Blot Analysis of Giloma

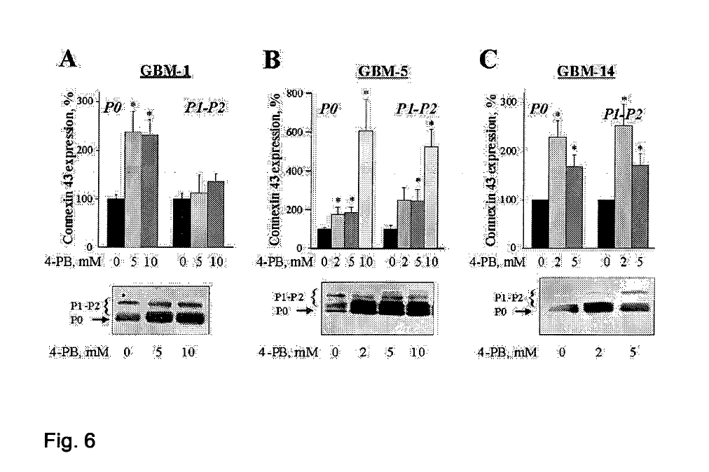

Primary glioblastoma cells were grown to 75% confluency and cultured for 48 hours in the presence or absence of 4-PB (2, 5 and 10 mM) and processed as previously described (Asklund et al, Exp cell Res, 2003, 284:183-193). Briefly, cell extracts, containing equal amount of protein (30 μg/lane for Cx43 detection and 60 μg/lane for GFAP detection) were loaded on 10% polyacrylamide gels, separated by SDS-PAGE, and electro-transferred onto nitrocellulose membranes. A rabbit polyclonal antibody to Cx43 diluted 1:8000 (Sigma) or a mouse monoclonal antibody to GFAP diluted 1:1000 (Pharmingen) were applied overnight at 40° C. The detection step was performed with the enhanced chemiluminescence (ECL) detection kit (Amersham). The membranes were exposed to Hyperfilm-ECL (Amersham) for 1-5 min. For quantification, films were scanned and analysed with Image Gauge (version 3.12, Fuji Photo Film Corp.). Data analysis was performed using the program Sigma Plot for Windows (version 6, Jandel Corp.). All results are expressed as means ± SE for the indicated number of experiments (FIG. 6).

Western Blot Analysis for Cx43 in Neural Stem Cells

Human neural stem cells (HNSC.100 cells, Villa et al Exp Neurol, 2000, 161(1):67-84) were obtained from Alberto Martinez Serrano, Department of Molecular Biology, Center of Molecular Biology Severo Ochoa, Autonomous University of Madrid-CSIC, Campus Cantoblanco, Madrid, 28049, Spain. Neural stem cells were grown to around 50% confluence and then 0.5 mM 4-PB treatment was started and continued for 4 days. Medium was replaced every 2^{nd} day with fresh drug. Whole-cell lysates from three 100 mm petri dishes were solubilized in 100 μl of lysis buffer containing 250 mM Tris-HCl (pH 8.5), 2% SDS, 10% glycerol, 0.05 mM PMSF and protease inhibitor cocktail (Roche). Protein concentrations were quantified using the micro BSA protein assay kit (Pierce). Cell lysates were further mixed with loading buffer containing 1% β-mercaptoethanol, 0.02% bromophenol blue, and 0.1 M DTT. Cell extracts, containing equal amounts of protein (50 μg/lane), were loaded onto 10% polyacrylamide gels, separated by SDS-PAGE, and electrically transferred onto nitrocellulose membranes (Schleicher & Schuell, Germany) with a transblot apparatus (Bio-Rad). For immunostaining, nonspecific binding was blocked for 30 min at room temperature with 5% nonfat dry milk dissolved in Tris-buffered saline supplemented with 0.05% Tween 20. A rabbit polyclonal antibody to Cx43 (1:8000 dilution in blocking buffer; Sigma) was applied overnight at 4° C. After three washes, membranes were exposed to a goat anti-rabbit secondary antibody coupled to horseradish peroxidase (1:3000 in blocking buffer; Bio-Rad). The detection step was performed with the enhanced chemiluminescence (ECL) detection kit (Amersham). The membranes were exposed to Hyperfilm-ECL (Amersham) for 1-5 min. For quantification, films were scanned and analyzed with Image Gauge (Version 3.12, Fuji Photo Film Corp.). All results are expressed as means ± SE for the indicated number of experiments (FIG. 13).

As an internal control for equal amount of protein loaded on the gel, the nitrocellulose membranes were stained with Ponceau S solution (0.2% Ponceau S, 3% trichloroacetic acid, and 3% sulfosalicylic acid) for 5 min. As the bands of protein became visible, the membranes were washed several times and total amount of loaded protein was quantified using computerized densitometric analysis.

Fluorescent Dye Transfer

Primary glioblastoma cells were cultured on 35 mm petri dishes and in vitro labelled with 5 μM calcein-AM (acetomethylic ester) and 10 μM Dil (Molecular Probes) diluted in supplemented serum free medium DMEM/F12 and incubated for 30 min at 37° C. The cells were washed once with the same medium, trypsinized and suspended in the culture medium induding 10% FBS. The cells were pelleted by centrifugation at 1600 rpm for 1 minute and resuspended in fresh culture medium. The procedure was repeated twice. Following the second resuspension, the labelled cells were plated on top of unlabelled cells of the same origin. These cells were either pretreated with 4-PB at a concentration of 5 mM, or non-treated for serving as a control. The labeled cells were then allowed to settle for 1 hour, followed by analyses using epifluorescence detection microscopy every 30 minutes for up to 6 hours. GJC (gap-junction communication) were studied using an Olympus BX50WI microscope equipped with UV epifluorescence and red and green attenuating filters.

Results:

Antiproliferative Effects of 4-Phenylbutyrate on Glioma Cells

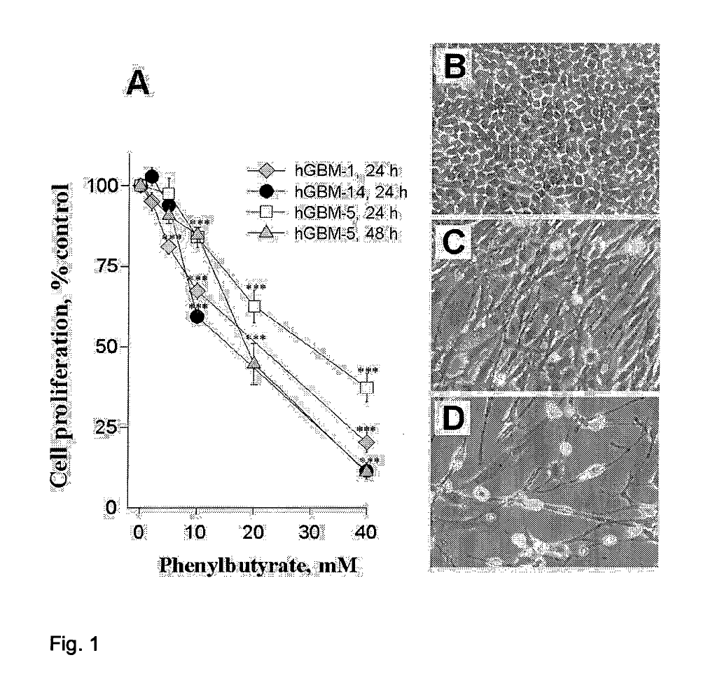

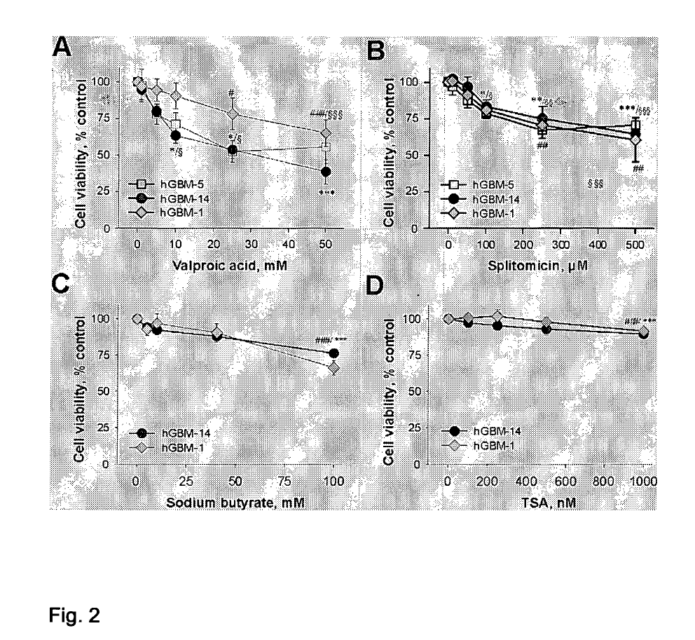

The three cell cultures, hGBM-1, hGBM-5 and hGBM-14, were treated with 4-PB at various concentrations and analyzed at different time points using the MTT-test and phase contrast microscopy. FIG. 1A shows the effect on cell proliferation in cultures treated at 2-20 mM. The hGBM-1 culture shows significant sensitivity to 4-PB already at 5 mM, while hGBM-5 and hGBM-14 are significantly affected at 10 mM 4-PB. In addition, prolonged treatments with a lower concentration (2 mM) were performed. FIG. 1B-D show phase contrast micrographs of hGBM-1 cells before treatment, at 1 day and 10 days of treatment, respectively. Anti-proliferative effects were seen already after 24 hours. In addition to 4-PB, other compounds were tested for their antiproliferative effects on these cell cultures. Splitomicin, Trichostatin A, valproic acid and sodium butyrate were all shown to be less potent, as analysed by MTT assay (FIG. 2).

Characterization of Cultured Tumour Cells

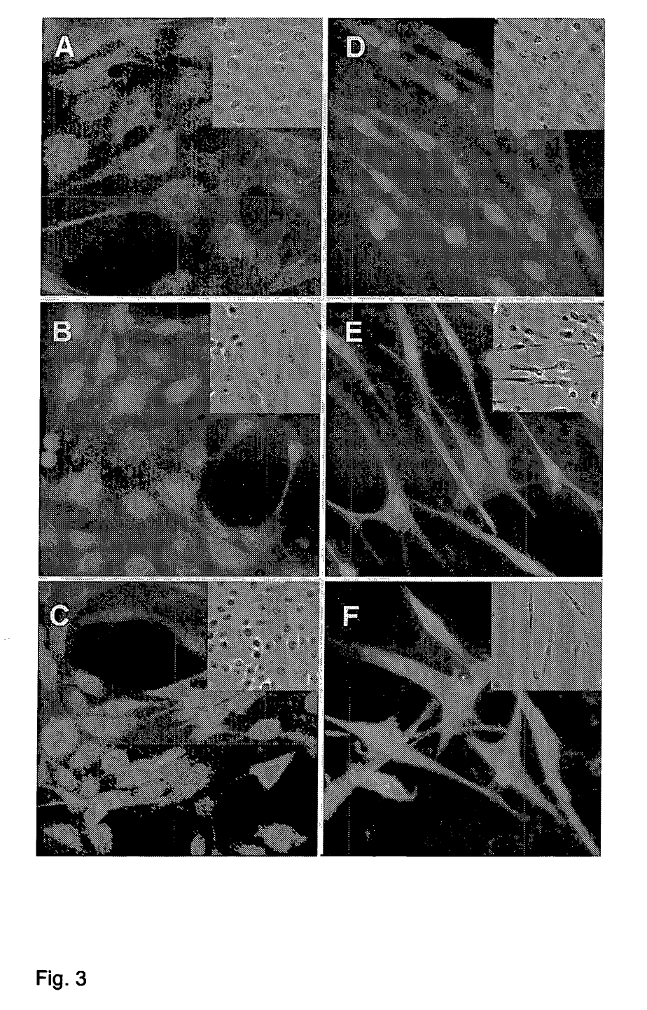

Tumour tissue specimens from patients undergoing surgical resection of a GBM were used to establish long-term primary cultures. The gross morphology of tumour cells varied between cultures but was quite consistent within each culture. The majority of cells were bipolar with short processes and identified as tumour cells of glial origin by their consistent expression of GFAP (FIG. 3A-C). Immunostainings for GFAP were done repeatedly over time in culture to verify maintenance of the glial phenotype of tumour cells. Selected cultures; hGBM-1, hGBM-5 and hGBM-14, remained stable with respect to morphology and expression of glial markers including GFAP, vimentin, and S-100 overtime in culture.

Effect of 4-Phenylbutyrate on Tumour Cell Morphology and GFAP Levels

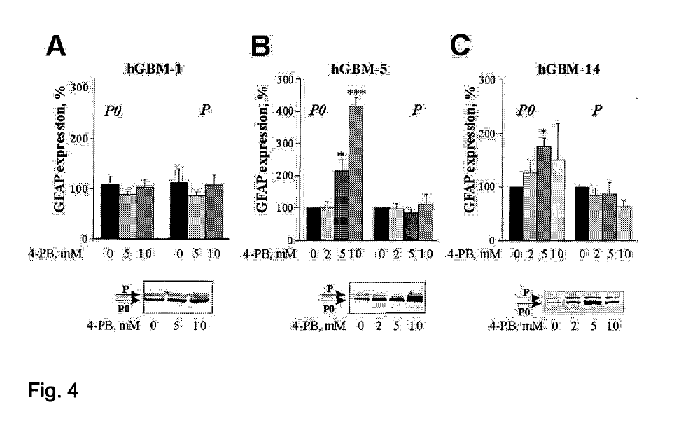

Semi-confluent glioblastoma cell cultures were incubated with 2 mM or 5 mM 4-PB in complete culture medium. Within the first 48 hours of treatment, the cells gradually changed their morphology to become more elongated with multiple fine cytoplasmic extensions (insets FIG. 3). This morphological transition was faster and more extensive in cultures exposed to 5 mM compared to 2 mM of 4-PB (data not shown). When analyzed for GFAP expression by immunocytochemistry, cells of all three cultures, hGBM-1, hGBM-5 and hGBM-14, showed a more intense immunostaining by epifluorescence detection after treatment with 4-PB. Moreover, cells of all three cultures showed a different subcellular distribution of GFAP immunoreactivity in the presence of 4-PB. In addition to the normal cytoplasmic pattern, GFAP was also associated with the nucleus and/or the nuclear membrane (FIG. 3D-F). The ratio of nuclear versus cytoplasmic GFAP immunoreactivity varied between treated cultures. Western blot analysis of GFAP revealed two distinct bands with slightly different apparent molecular weight (FIG. 4). These bands were interpreted to represent phosphorylated and non-phosphorylated isoforms of GFAP. 4-PB treatment specifically upregulated the non-phosphorylated isoform of GFAP in two out of the three human glioblastoma cell cultures, hGBM-5 and hGBM-14 (FIG. 4.), while the level of the phosphorylated isoform remained stable.

4-Phenylbutyrate Increases Protein Levels of Connexin 43 and Induces its Subcellular Redistribution

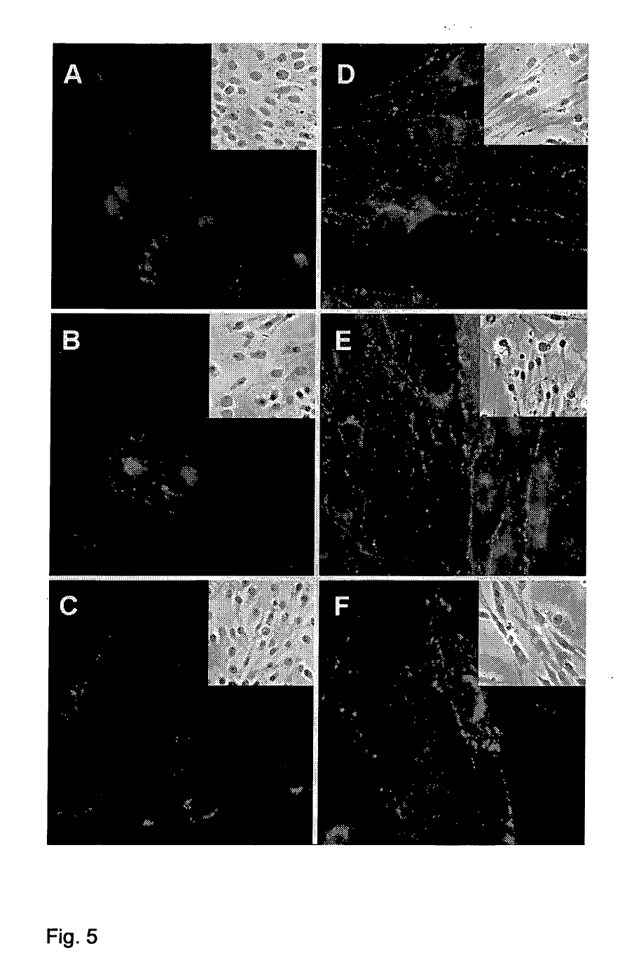

The gap junction protein Cx43 was detected in all three primary cultures by immunocytochemistry and by Western blot analysis using a commercially available polyclonal anti-connexin 43 antibody. Immunocytochemistry demonstrated the presence of Cx43 in the plasma membrane as patchy cell surface fluorescence with a finely granular appearance (FIG. 5). Following treatment with 5 mM 4-PB for 48 hours, cells showed a marked increase of Cx43 immunoreactivity at the cell surface. The density of Cx43 immunoreactivity varied between cells, but there was now a distinct lateralization of Cx43 immunreactivity with preference to areas of intercellular contacts and cellular processes (FIG. 5).

Western blot analysis of whole-cell extracts detected three immunoreactive isoforms of Cx43. These isoforms differed slightly by apparent molecular weight and migrated as a smaller non-phosphorylated isoform, and bigger phosphorylated Cx43 isoforms (FIG. 6). Following treatment with 4-PB (2-10 mM), both non-phosphorylated and phosphorylated isoforms of Cx43 increased quantitatively in glioma cells (FIG. 6). Similarly, the level of Cx43 protein increased in human neural stem cells following treatment with 0.5 mM 4-PB (FIG. 13).

4-Phenylbutyrate Enhances Gap Junction Communication

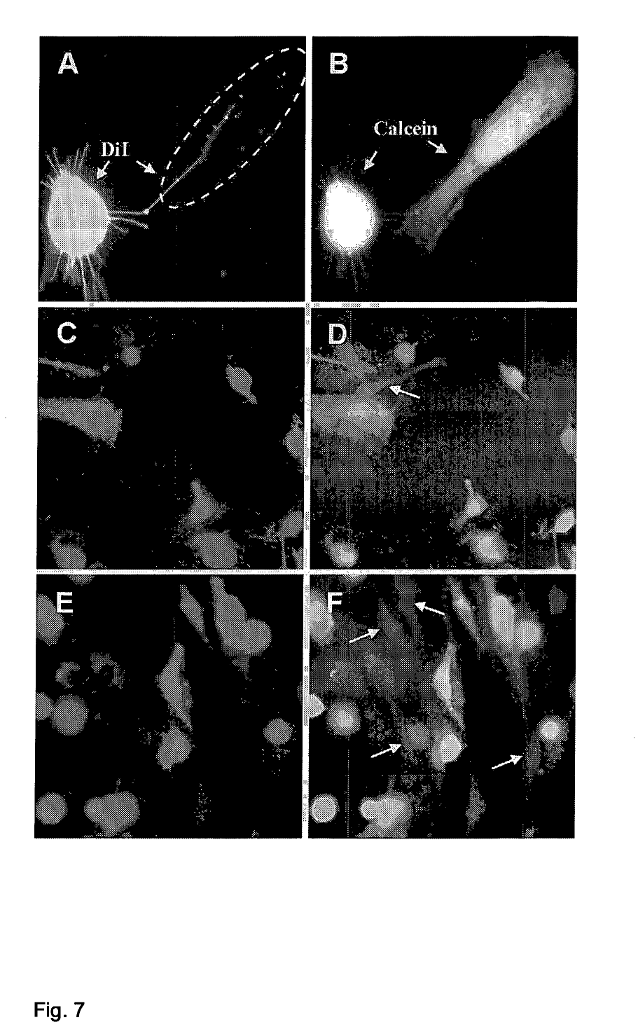

In order to study GJC in the glioma cultures, the cells were labelled with two fluorescent dyes, Dil and Calcein. Cells were trypsinized, washed and co-plated with unlabelled cells of the same origin cultured and adherent to a petri dish. Within one hour, preloaded cells had settled and attached to the subconfluent monolayer of unlabelled cells. Since calcein is a water-soluble aceto-methylic ester which appear green after intracellular esterase cleavage, green fluorescence in Dil-negative cells must originate from pre-loaded donor cells indicative of functional contacts between labeled and unlabeled tumour cells. This dye transfer was mediated by gap junctions located on very thin and slender processes projecting from dye loaded donor cells (FIGS. 7A and 7B). When recipient cells were cultured in the presence of 5 mM 4-PB for 48 hours prior to dye transfer, the intercellular spread of calcein was markedly improved compared to untreated cells (FIG. 7).

### Example 2

### Enhancement of Gap Junction-Mediated Intercellular Communication and Bystander Effect by 4-PB.

Materials and Methods

Tissue Processing and Culture Maintenance

GBM primary cultures were established from surgical tumour biopsies and characterized by immunocytochemistry and Western blot analysis as previously described (Asklund et al (2003) Exp. Cell Res., 284: 183-193.). Glioblastoma cells were cultured in DMEM/F12 culture medium supplemented with 10% FCS (Gibco), penicillin/streptomycin (100 U/mI, Gibco).

Gap Junction Mediated Cell Death

Glioma cells with stable expression of green fluorescent protein (EGFP) and the herpes simplex virus thymidine kinase expressing gene HSV-TK in sense (S6) or in antisense (A1) were mixed with cells stably expressing the red fluorescent protein (RFP) (ratio 3:7 S6:RFP or A1:RFP). Ganciclovir, 4-phenylbutyrate and AGA were added 3 hrs after seeding the cells, except for AGA, which was added directly at seeding of cells. After 48 hours, cells were split and after 96, 120 and 168 hrs one each of the cell culture quadruplicates was used for MTT-test while one sample was employed for FACS analysis. After 144 h cells from the third of the quadruplicate were split again and the last quadruplicate sample was analyzed the following day (168 h).

Assessment of Cell Viability and Cell Proliferation by MTT Assay

Proliferation of cultured glioma cells was assessed by 3-(4,5-dimethylthiazol-2-yl)-2,5-diphenyltetrazolium bromide (MTT, Sigma) assay. MTT tetrazolium salt was dissolved in serum-free culture medium (0.5 mg/ml), added to the cells (150 μl/well) and incubated for 30 min at 37° C. Formazan dye, formed by viable cells, was solubilized in 300 μl of isopropanol. Aliquots (100 μl) of the solutions were transferred to 96-well microplates. The absorbance at 570 nm (with reference at 650 nm) was measured using a microtiter plate spectrophotometer (Anthos HT III). The results were expressed as percentage of viable cells compared to the control sample of untreated cells (100%).

Fluorescent Dye Transfer between Glioma Cells

Glioblastoma cells were cultured on 35 mm petri dishes and in vitro labelled with 5 μM calcein-AM (acetomethylic ester) and 10 μM Dil (Molecular Probes) diluted in supplemented serum free medium DMEM/F12 and incubated for 30 min at 37° C. The cells were washed once with the same medium, trypsinized and suspended in the culture medium induding 10% FBS. The cells were pelleted by centrifugation at 1600 rpm for 1 minute and resuspended in fresh culture medium. The procedure was repeated twice. Following the second resuspension, the labelled cells were plated on top of unlabelled cells of the same origin. These cells were either pretreated with 4-PB at a concentration of 5 mM, or non-treated serving as a control. The labeled cells were then allowed to settle for 1 hour, followed by analyses using epifluorescence detection microscopy every 30 minutes for up to 6 hours. GJC were studied using an Olympus BX50WI microscope equipped with UV epifluorescence and red and green attenuating filters.

Fluorescent Dye Transfer between Neural Stem Cells and Glioma Cells

Donor human neural stem cells (1×10⁵ HNSC100) and recipient human glioblastoma cells (1×10⁵ U343MGa) cells were plated each in 4 separate 35 mm petri dishes. At about 70% confluency, 2 plates of donor cells were treated with 0.5 mM 4-PB and 2 plates of recipient cells were treated with 5 mM PB and 65 μM AGA (18α glycyrrhethinic acid dissolved in DMSO; Sigma) both for 24 hours. Donor cells were incubated with 10 μM Dil (a fluorescent lipophilic carbocyanine tracer; Molecular Probes) and 5 μM Calcein-AM (an acetoxymethyl ester derivative; Molecular Probes) for 20 min in complete medium. Cells were washed three times with culture medium and twice with PBS to remove free dye. After trypsinization, cells were pelleted by centrifugation and resuspended in co-culture medium. Donor cells 50% confluent were added to the plate of recipient cells. AGA treatment, when performed, was always continued throughout the experiment. Assessment of functional GJC by Calcein dye transfer from donor to recipient cells was carried out after 4 hours using an Olympus BX50WI microscope.

Results

4-PB Upregulates Gap Junction Protein Connexin 43 Expression in Glioblastoma Cells.

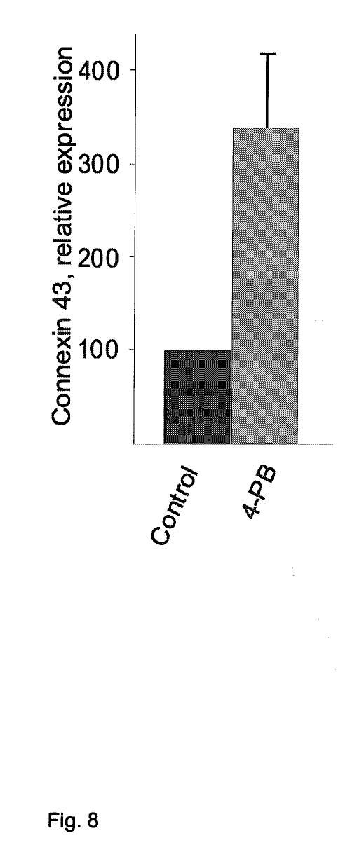

Treatment of glioblastoma cell cultures with 2 mM 4-PB for 2 days resulted in increased expression of the intermediate filament protein and differentiation marker Glial Fibrillary Acidic Protein (GFAP) and the gap junction protein connexin 43 (FIG. 8).

Conjunctional Treatment of Glioma Cells with 4-Phenylbutyrate and Ganciclovir Facilitates Bystander-Mediated Cellkilling.

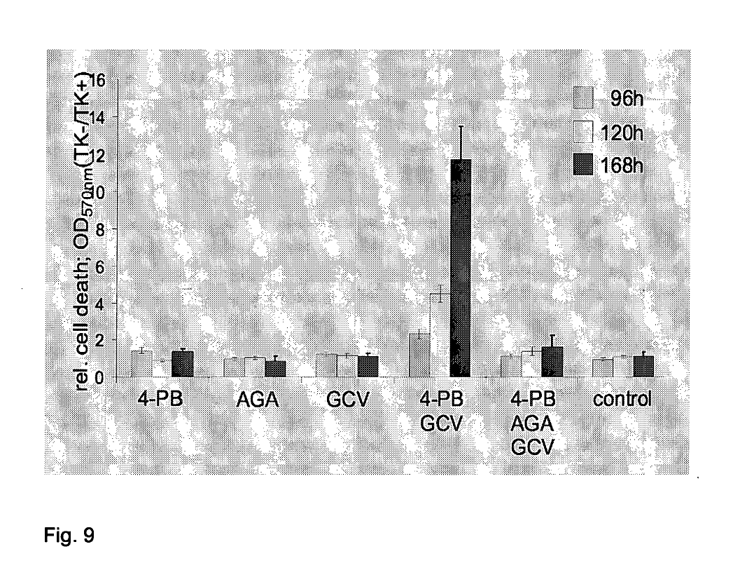

Rat glioma cells expressing the HSV-TK gene in sense (S6) and antisense (A1) orientation were mixed with RFP expressing (HSV-TK negative) cells, in a ratio of 30% HSV-TK and 70% RFP cells. To investigate specific effects of combinding drug treatments, ratios from results of MTT analysis from co-cultured clones A1-RFP (HSV-TK negative) over S6-RFP (HSV-TK positive) were calculated. These ratios show a time dependent increase in cell death of cell populations with HSV-TK positive cells when ganciclovir and 4-PB are combined. The bystander killing effect was reduced by addition of a-glycyrrhetinic acid (AGA), an inhibitor of gap junction communication (FIG. 9).

Fluorescent Dye Transfer between Glioma Cells through Gap Junction Communication

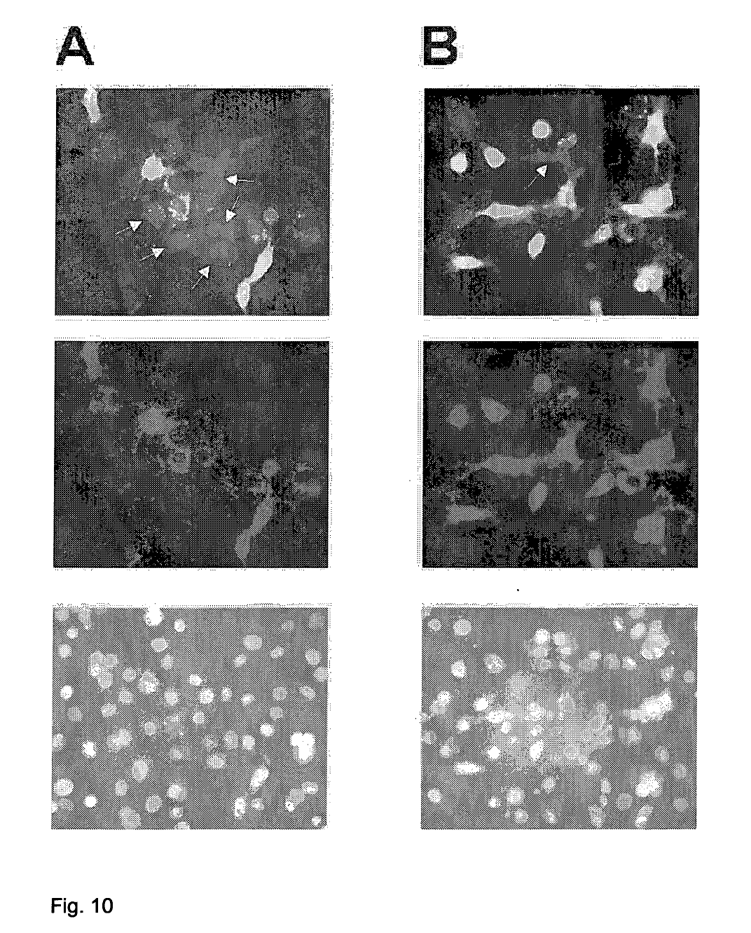

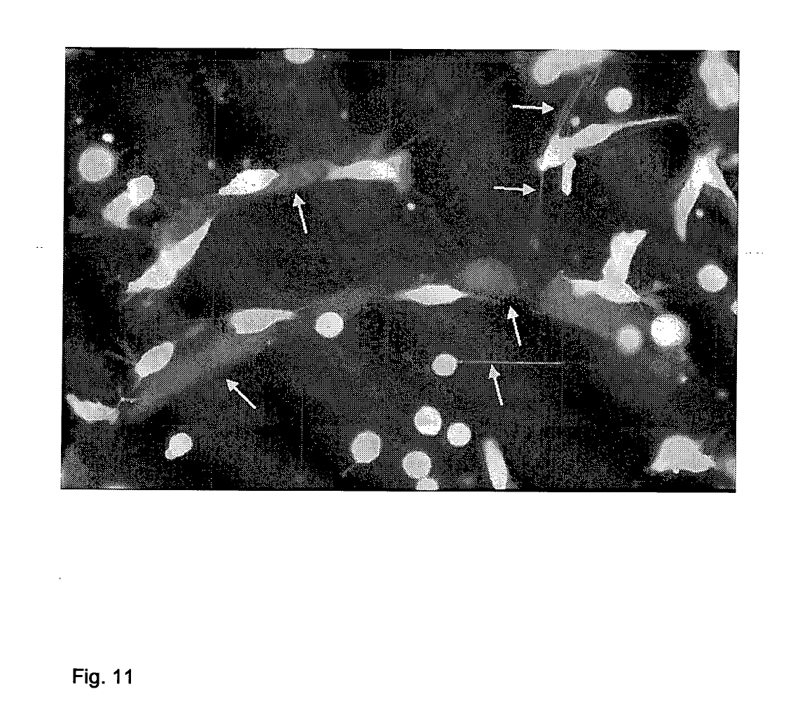

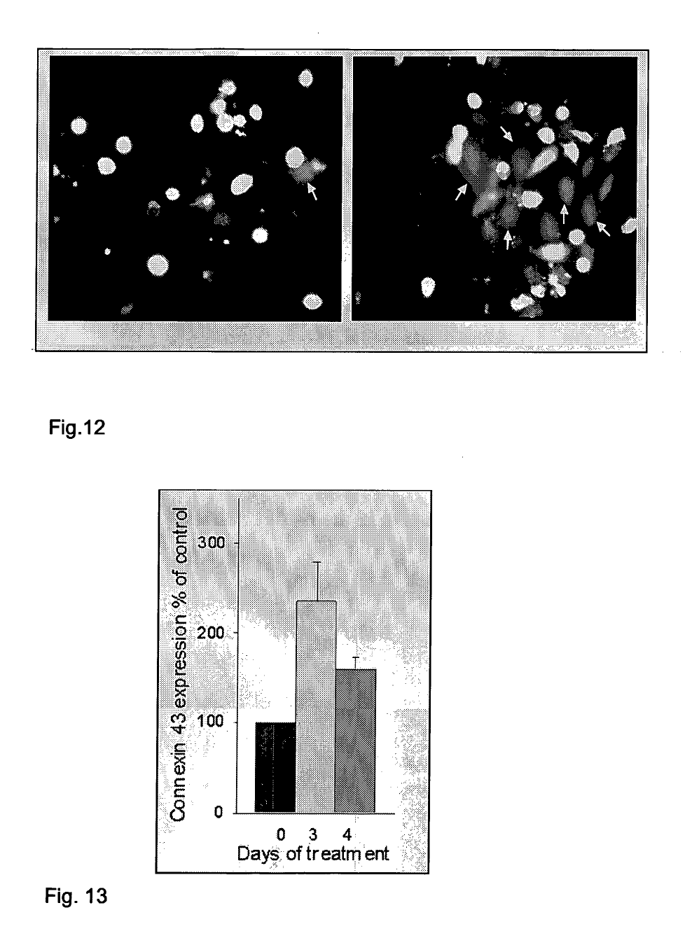

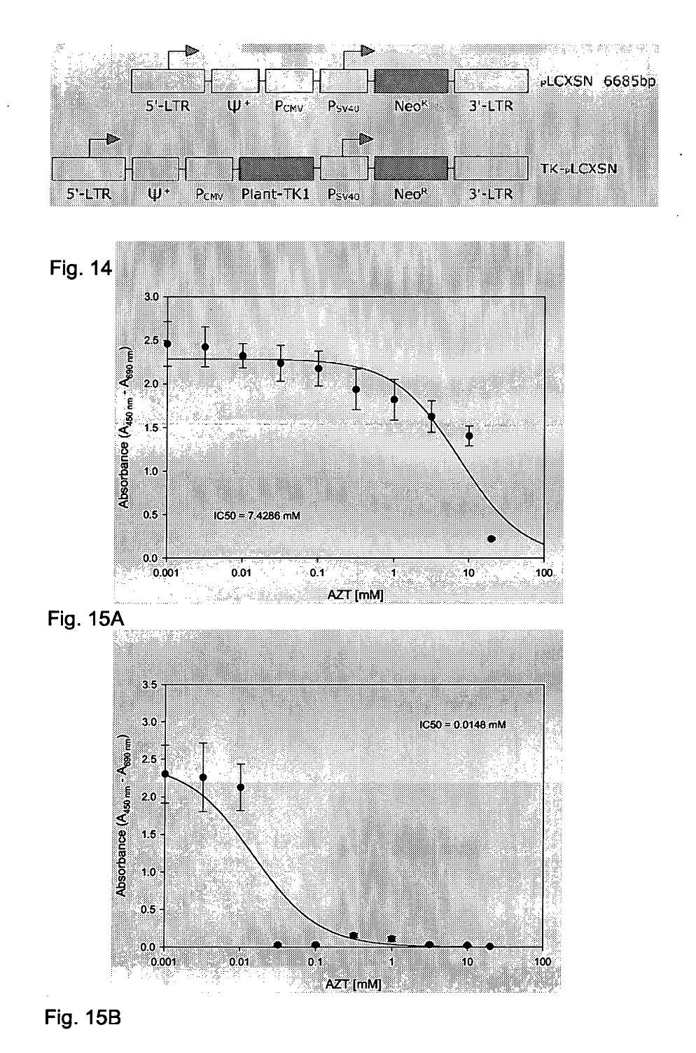

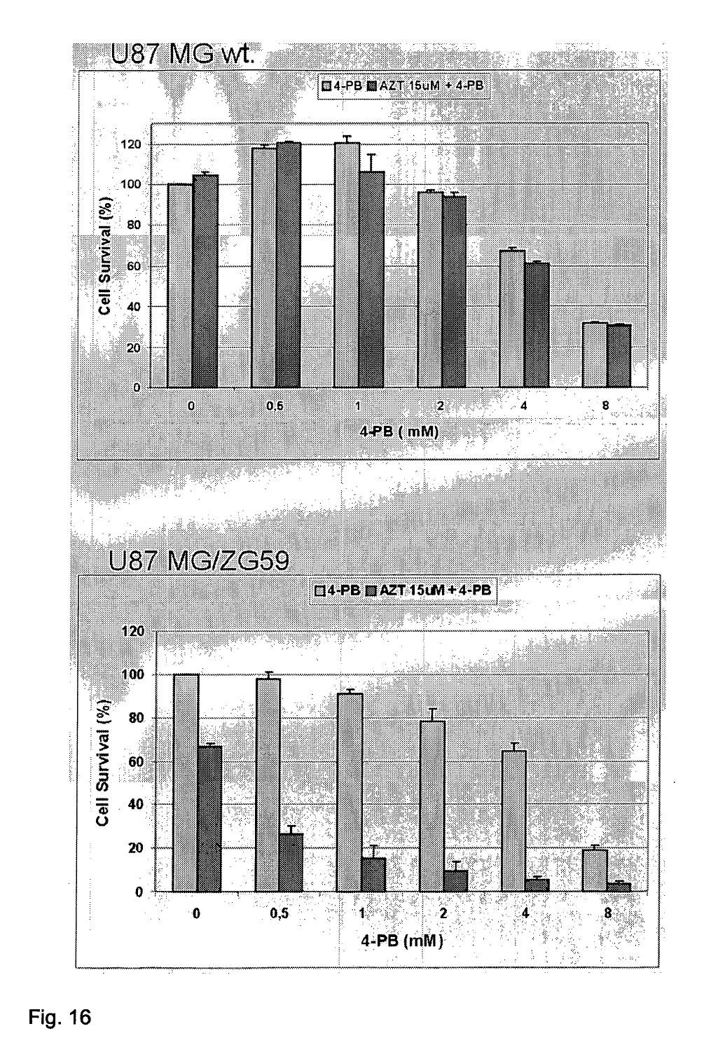

Glioblastoma cells (S6 cells) preloaded with calcein and Dil, as described above, were mixed with unlabeled recipient cells. Functional gap junction communication was assessed by the efficient transfer of calcein from Dil-positive donor cells. The transfer could be inhibited by the gap junction inhibitor AGA (FIG. 10).

Gap Junction Communication between Human Neural Stem Cells and Human Glioblastoma Cells.

Dye transfer between human neural stem cells (HNSC-100) and human glioma cells was observed and assessed semi-quantitatively by fluorescent dye transfer technique. Stem cells were preloaded with fluorescent dye, as described above. Cells were mixed in a ratio of approximately. 30% stem cells with 70% glioma cells. Functional gap junction coupling was analysed using epifluorescence microscopy (FIG. 11). Efficient dye transfer can be seen from the preloaded stem cells cells (yellow) to the glioblastoma cells (green).

Pre-treatment of the neural stem cells and glioblastoma cells for 48 hours with 4-PB, 0.5 mM and 2 mM respectively, substantially enhanced the dye transfer as compared to untreated cells (FIG. 12).

In addition to the 4-PB induced expression of connexin 43 in glioblastoma cell cultures (FIG. 8), this drug also upregulated connexin 43 expression in human neural stem cells (FIG. 13).

Conclusions

Gap junction communication can mediate transfer of low molecular weight (<1000 Da) compounds from one cell to another. This can be exploited in a cell-mediated suicide gene paradigm with transfer of cytotoxic molecules from one cell to another. The therapeutic efficacy is related firstly to the delivery of suicide gene-expressing cells to the site of disease, secondly to the efficient conversion of a non-toxic prodrug to a cytotoxic compound within the recombinant cells carrying the suicide gene, and thirdly to the transfer of cytotoxic drug to neighbouring cells (bystander effect). We strongly believe that short chain-aromatic fatty acids, here exemplified by 4-PB, can facilitate the communication between cells by up-regulating gap junction structure and function.

Targeting of glioblastoma cells in vivo will be done by using human neural stem cells, which previously have been shown to migrate to, and identify glioblastoma cells, in the brain tissue. Our data suggest that stem cells expressing suicide genes, combined with short chain aromatic fatty acids to increase gap junction communication, can potentiate the activation and spread of systemically delivered prodrug to make this tumour cell eradication strategy clinically applicable.

### Example 3

### 4-PB Enhances the Cytotoxic Effect of Nucleoside Analogues

4-Phenylbutyrate Cytotoxicity Assay

Briefly, cells were harvested by trypsinzation, stained by Tryphan Blue for cell viability counting, seeded in triplicate in 96-well plates (1.000-2.000 cells/well) where after media conditioned with a fixed concentration of phenyl butyrate (3.16 mM) were added to the plates followed by incubation for 24 hours at 37° C. and 5% CO₂. Cell proliferation/cytotoxicity was assessed by adding the colour generating substrates of the Cell Proliferation kit II (XTT) from Roche (Cat. No. 1465015) and measuring absorbance at 450 nm and 690 nm with a Thermo Labsystems Multiscan Ascent reader.

Pre-Incubation of 4-Phenyl Butyrate Prior to Cytotoxicity Effect Assay

Cells were seeded at a density of 1.000-2.000 cells/well in 96-well plates. Cells were pre-incubated with 4-phenyl butyrate for 24 hours prior to drug exposure by adding media conditioned with phenyl butyrate at a fixed concentration (3.16 mM). Plated incubated for 24 hours (37° C., 5% CO₂) where after media was discarded and replaced by media conditioned with various concentrations of nucleoside analogs (e.g. AZT), 200 μl/well. After 120 hours of drug exposure the experiments were terminated. Cell proliferation/cytotoxicity was assessed by adding the colour generating substrates of the Cell Proliferation kit II (XTT) from Roche (Cat. No. 1465015) and measuring absorbance at 450 nm and 690 nm with a Thermo Labsystems Multiscan Ascent reader.

The data was corrected for background media-only absorbance where after the 50% growth inhibitory concentration—IC₅₀ (the dose that inhibits cell growth by 50%) was calculated for each treatment.

Results

U343MG cells pre-incubated (24 h) w/Phenylbutyrate:

4.2 times AZT sensitivity improvement
   CL2:6 cells pre-incubated (24 hours) w/Phenylbutyrate
1.7 times AZT sensitivity improvement after 120 hours of drug exposure.
1.4 times GCV sensitivity increase after 120 hours of drug exposure
   U87MG cells pre-incubated (24 h) w/Phenylbutyrate:
3.5 times AraC sensitivity increase after 120 hours of drug exposure
2.3 times CdA sensitivity increase after 120 hours of drug exposure

7.8 times Gemcitabine sensitivity increase after 120 hours of drug exposure

| IC₅₀ | U343MG | CL2:6 | U87MG | |
|---|---|---|---|---|
| PB | | | 4.139 | mM |
| AZT | 0.056 mM | 0.573 mM | | |
| PB + AZT | 0.0133 mM | 0.349 mM | | |
| GCV | 0.1044 mM | 3.356 mM | | |
| PB + GCV | 0.0799 mM | 2.341 mM | | |
| AraC | | | 4.335 | mM |
| AraC + PB | | | 1.253 | mM |
| CdA | | | 5.735 | μM |
| CdA + PB | | | 2.508 | μM |
| Gemcitabine | | | 0.2478 | μM |
| Gemcitabine + PB | | | 0.03166 | μM |

Conclusion.

4-PB clearly enhanced the efficacy of a series of well-known nucleoside analogues even in the absence of a heterologous deoxynucleoside kinase.

### Example 3b

### 4-Phenylbutyrate (4-PB)—Trl Butyrate, Synergistic Effect with Nucleoside Analogs.

Materials and Methods

Cell Culture

The human Glioblastoma cell line U-87-MG (ATCC, HTB-14) was cultured in EMEM (Cambrex) conditioned with 10% Foetal Bovine Serum (FBS) (Gibco) and Gentamicin 1 ml/L (Cambrex). U343MG and CL2:6 cells (from Karolinska Institute) were cultured in DMEM (Cambrex) conditioned with 10% Foetal Bovine Serum (FBS) (Gibco) and Gentamicin 1 ml/L (Cambrex). Cells were grown at 37° C. and 5% CO₂ in a humidified incubator.

4-Phenylbutyrate Cytotoxicity Assay

Briefly, cells were harvested by trypsinzation, stained by Tryphan Blue (Cambrex) for cell viability counting, seeded in triplicate in poly-L-lysine (PLL) (Sigma # P6282) coated 96-well plates (1.000-2.000 cells/well) where after media conditioned with a fixed concentration of phenyl butyrate (3.16 mM) was added to the plates followed by incubation for 24 hours at 37° C. and 5% CO₂. Cell proliferation/cytotoxicity was assessed by adding the colour generating substrates of the

Cell Proliferation kit II (XTT) from Roche (Cat. No. 1465015) and measuring absorbance at 450 nm and 690 nm with a Thermo Labsystems Multiscan Ascent reader.

Pre-Incubation of 4-Phenyl Butyrate Prior to Cytotoxicity Effect Assay

Cells were seeded at a density of 1.000-2.000 cells/well in PLL coated 96-well plates. Cells were pre-incubated with 4-phenyl butyrate for 24 hours prior to drug exposure by adding media conditioned with phenyl butyrate at a fixed concentration (3.16 mM). Plated incubated for 24 hours (37° C., 5% CO₂) where after media was discarded and replaced by media conditioned with various concentrations of nucleoside analogs; AZT (Sigma # A2169), AraC (Sigma # C6645), Cladribine (CdA) (Sigma #C4438), Gancidovir (GCV) (Sigma # G2536) and Gemcitabine (Orifarm), 200 μl/well. After 120 hours of drug exposure the experiments were terminated. Cell proliferation/cytotoxicity was assessed by adding the colour generating substrates of the Cell Proliferation kit II (XTT) from Roche (Cat. No. 1465015) and measuring absorbance at 450 nm and 690 nm with a Thermo Labsystems Multiscan Ascent reader. he data was corrected for background media-only absorbance where after the 50% growth inhibitory concentration—IC₅₀ (the dose that inhibits cell growth by 50%) was calculated for ach treatment.

Consfruction of Retrovirus Vectors and Transduction Procedure

Mid scale production and concentration of Moloney Murine Leukemea Virus (MMLV)-derived replication defective VSV-G pseudo-typed retroviruses, typically yielding a total of 10⁷ transducing units, was performed in 293 T cells. The 293T packaging cells (ATTC CRL-11268) were cultured at 37° C., 5% CO₂ in OPTIMEM 1 medium (Life Technologies, Inc.).

The constructed pLCXSN (Clontech) plasmid vector pZG59 (coding for a Tomato Thymidine kinase I with a C-terminal deletion of 26 amino acids, WO 03/100045) and pVPack-GP (Stratagene) plus pVPack-VSV-G (Stratagene) were transfected into the packaging cells using LipofectAMINE PLUS (Invitrogen—Life Technologies, Inc.) according to the protocol provided by the supplier. The medium from the transfected cells, cultured in DMEM (Invitrogen), was collected 48 and 72 hours post transfection, filtered through a 0.45 μm filter, pelleted by ultracentrifugation (50.000 g, 90 min at 4° C.) and dissolved in DMEM (Invitrogen). The titer of the virus was determined by reverse transcriptase assay. The virus was subsequently used to transduce the cancer cell lines.

Retro Viral Transduction

The day before transduction, 5×10⁴ cells/well were seeded in 24-well plates. On the day of transduction, viruses were added with a MOI of 5 with polybrene (5 μg/ml media). Cells incubated for 3 hours and thereafter media was renewed and cells were expanded and selected by addition of Geneticin® (G 418, Invitrogen—Life Technologies, Inc.) for 14 day. Subsequently cells were ready for experiments.

Cell Killing Effect of AZT in U87MG/Tomato Kinase Positive Cells

Exponentially growing U87 wt and tomato kinase expressing cells were plated at a density of 2.000 cells/well in poly-L-lysine coated 96-well plates in 100 μl conditioned medium and incubated 37° C. in a humidified incubator with a gas phase of 5% CO₂. After 24 hours, medium was replaced with medium containing varying concentrations of AZT. Hereafter, cells were exposed to drug conditioned media for 120 constitutive hours. The chemo resistance of cultured cells was monitored by the surviving cell fraction as a function of the drug concentration. Viability of cells was determined via the colorimetric XTT assay (XTT kit II—Roche, cat no. 1465015). Briefly, cell media was carefully removed and 100 μl fresh media and 50 μl XTT mix was added to each well. The absorbance at 450 and 690 nm was determined using an ELISA plate reader (Ascent, ThermoLab). The IC₅₀ value (50% inhibition concentration) of the investigated compound was calculated as the mean value of each experiment using SigmaPlot® (Dyrberg Trading, DK).

Synergistic Cell Killing Effect of AZT and 4-PB in U87MG/Tomato Kinase Positive Cells

U87MG/tk and U87MG wt. cells were plated in 12-well plates (40.000 celIs/well) containing 1 ml of medium (EMEM, Cambrex) with 10% fetal bovine serum (Invitrogen). After 24 hours of culturing, treatment was started with 15 μM AZT with or without combination of different concentrations of 4-phenylbutyrate (0.5, 1,2,4,8 mM) and treatment was continued for 72 hours. Medium was replaced by 300 μL of MTT solution (0.5mg/mL; Sigma # M 2128) per well and incubated at 37° C. for 30 minutes. The MTT solution was aspirated and 350 μL of isopropranol was added to dissolve the formazan product by gentle shaking. Three 100 μL aliquots of formazan solution from each well were pipetted into a 96-well microplate. Optical density was measured by Multiscan RMCC/340 plate reader (MicroELISA Reader) at 570 nm with reference at 650 nm. The results were expressed as percentage of live cells compared to control untreated cells (100%). The analyses were run as three independent experiments.

Results

U343MG cells pre-incubated (24 h) w/Phenylbutyrate demonstrated a 4.2 times AZT sensitivity improvement whereas CL2:6 cells showed a 1.7 times sensitivity increase for AZT and 1.4 times for GCV after 120 hours of drug exposure

In the U87MG cells pre-incubated (24 h) w/Phenylbutyrate sensitivity increased 3.5 times for AraC, 2.3 times for CdA and 7.8 times for Gemcitabine after 120 hours of drug exposure

| IC₅₀ | U343MG | CL2:6 | U87MG | |
|---|---|---|---|---|
| AZT | 0.056 mM | 0.573 mM | 5.999 | mM |
| 4-PB + AZT | 0.0133 mM | 0.349 mM | 2.329 | mM |
| GCV | 0.1044 mM | 3.356 mM | | |
| 4-PB + GCV | 0.0799 mM | 2.341 mM | | |
| AraC | | | 4.335 | mM |
| 4-PB + AraC | | | 1.253 | mM |
| CdA | | | 5.735 | μM |
| 4-PB + CdA | | | 2.508 | μM |
| Gemcitabine | | | 0.2478 | μM |
| 4-PB + Gemcitabine | | | 0.03166 | μM |

Cell killing by AZT of tomato kinase expressing U87MG

The tomato thymidine kinases expressing U87 cells demonstrated a 500- fold sensitivity increase when compared to the wt U87MG cells (0.0148 mM vs. 7.4286 mM, respectively) (FIG. 15A vs 15B).

Enhanced Cell Killing by 4-PB and AZT Combination on Tomato Kinase Expressing Glioma Cells

In experiments running for 72 hours, 4-PB enhanced the effects of AZT in U87MG cells expressing the tomato kinase. Already at concentrations of 15 μM AZT and 0.5 mM 4-PB clear additive or synergistic effects were observed (FIG. 16). 15 μM AZT corresponds approximately to the IC50 measured in FIG. 15B.

Conclusion.

4-PB clearly enhanced the efficacy of a series of well-known nucleoside analogues even in the absence of a heterologous deoxynucleoside kinase. 4-PB also increased the cell killing efficiency of tomato kinase+nucleoside analogue in a synergistic way.

1. A pharmaceutical composition comprising at least one compound capable of enhancing gap-junction communication and at least one nucleoside analogue.

2. The composition of claim 1, wherein said compound capable of enhancing gap-junction communication is an aromatic organic acid, a pharmaceutically acceptable salt or an ester or amide of said acid.

3. The composition of claim 2, wherein said aromatic organic acid is a compound of the formula: 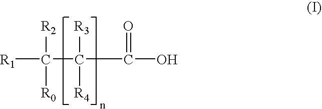
wherein R₀ is aryl (e.g., phenyl, napthyl), phenoxy, substituted aryl (e.g., one or more halogen [e.g., F, Cl, Br, I], lower alkyl [e.g., methyl, ethyl, propyl, butyl] or hydroxy substituents) or substituted phenoxy (e.g., one or more halogen [e.g., F, Cl, Br, I], lower alkyl [e.g., methyl, ethyl, propyl, butyl] or hydroxy substituents);
R₁ and R₂ are each H, lower alkoxy (e.g., methoxy, ethoxy), lower straight and branched chain alkyl (e.g., methyl, ethyl, propyl, butyl) or halogen (e.g., F, Cl, Br, I);
R₃ and R₄ are each H, lower straight and branched chain alkyl (e.g., methyl, ethyl, propyl, butyl), lower alkoxy (e.g., methoxy, ethoxy) or halogen (e.g., F, Cl, Br, I); and n is an integer from 0 to 2;
salts thereof (e.g., Na⁺, K⁺ or other pharmaceutically acceptable salts); stereoisomers thereof; and mixtures thereof.

4. The composition of claim 2, wherein R₀=aryl, phenoxy, substituted aryl or substituted phenoxy;
R₁ and R₂=H, lower alkoxy, lower straight and branched chain alkyl or halogen;
R₃ and R₄=H, lower alkoxy, lower straight and branched chain alkyl or halogen; and n=an integer from 0 to 2;
salts thereof; stereoisomers thereof; and mixtures thereof.

5. The composition of claim 3, wherein said aromatic fatty acid is selected from the group consisting of phenylacetic acid, phenylpropionic acid, phenylbutyric acid, 1-naphthylacetic acid, phenoxyacetic acid, phenoxypropionic acid, phenoxybutyric acid, 4-chlorophenylacetic acid, 4-chlorophenylbutyric acid, 4-iodophenylacetic acid, 4-iodophenylbutyric acid, α-methylphenylacetic acid, α-methoxyphenylacetic acid, α-ethylphenylacetic acid, α-hydroxyphenylacetic acid, 4-fluorophenylacetic acid, 4-fluorophenylbutyric acid, 2-methylphenylacetic acid, 3-methylphenylacetic acid, 4-methylphenylacetic acid, 3-chlorophenylacetic acid, 3-chlorophenylbutyric acid, 2-chlorophenylacetic acid, 2-chlorophenylbutyric acid and 2,6-dichlorophenylacetic acid, and the sodium salts of the these compounds.

6. The composition of claim 5, wherein the aromatic organic acid is 4-Phenylbutyrate or a pharmaceutically acceptable prodrug thereof.

7. The composition of claim 2, wherein the aromatic organic acid is 2-Phenylbutyrate or a pharmaceutically acceptable prodrug thereof.

8. The composition of claim 5, wherein the aromatic organic acid is phenylacetic acid or a pharmaceutically acceptable salt or an ester of phenylacetic acid.

9. The composition of claim 1, wherein the compound capable of enhancing gap-junction communication is valproic acid, a pharmaceutically acceptable salt thereof or a prodrug of valproic acid.

10. The composition of claim 1, wherein the compound capable of enhancing gap-junction communication is splitomicin, a pharmaceutically acceptable salt thereof or a prodrug of splitomicin.

11. The composition of claim 1, wherein the compound capable of enhancing gap-junction communication is butyric acid, a pharmaceutically acceptable salt thereof or a prodrug of butyric acid.

12. The composition of claim 1, further comprising a source of deoxyribonucleoside kinase.

13. The composition of claim 12, wherein the source of deoxyribonucleoside kinase is a gene therapy vector.

14. The composition of claim 13, wherein the gene therapy vector is a virus vector.

15. The composition of claim 14, wherein the virus vector is selected from the group consisting of being a viral vector, in particular a Herpes simplex viral vector, an adenoviral vector, an adenovirus-associated viral vector, a lentivirus vector, a retroviral vector or a vacciniaviral vector.

16. The composition of claim 15, wherein the source of deoxyribonucleoside kinase comprises a composition of packaging cells capable of producing an infective virion comprising said virus vector.

17. The composition of claim 13, wherein the gene therapy vector is a plasmid vector.

18. The composition of claim 17, wherein the plasmid vector is selected from the group consisting of general eukaryotic expression vectors, vectors for stable and transient expression and epitag vectors as well as their TOPO derivatives for fast cloning of desired inserts.

19. The composition of claim 12, wherein the source of deoxyribonucleoside kinase comprises a protein formulation.

20. The composition of claim 19, wherein the protein is formulated as a liposome composition.

21. The composition of claim 12, wherein the source of deoxyribonucleoside kinase comprises a composition of human stem cells genetically engineered to express a heterologous deoxyribonucleoside kinase.

22. The composition of claim 21, wherein the stem cells used in a stem cell-mediated therapy approach originates from the same tissue as the tumor cells or the same growth layer or alternatively originates from the bone marrow.

23. The composition of claim 12, wherein the source of deoxyribonucleoside kinase comprises a composition of human progenitor or precursor cells genetically engineered to express a heterologous deoxyribonucleoside kinase.

24. The composition according to claim 12, wherein the deoxyribonucleoside kinase is selected from the group consisting of
a. a deoxyribonucleoside kinase having the amino acid sequence of any of SEQ ID No 1 to 17;
b. a deoxyribonucleoside kinase variant comprising an amino acid sequence having at least 70% sequence identity to any of SEQ ID No 1 to 17; and
c. a deoxyribonucleoside kinase encoded by a nucleotide sequence capable of hybridising under conditions of high stringency to a nucleotide sequence encoding any of SEQ ID No 1 to 17.

25. The composition of claim 24, wherein the deoxyribonucleoside kinases comprises a deoxyribonucleoside kinase selected from the group consisting of
a. a deoxyribonucleoside kinase having the amino acid sequence of any of SEQ ID NO 1 to 5; and
b. a deoxyribonucleoside kinase variant comprising an amino acid sequence having at least 70% sequence identity to any of SEQ ID No 1 to 5 and having dNK activity.

26. The composition according to claim 1, wherein the nucleoside analogue is selected from the group consisting of aciclovir (9-[2-hydroxy-ethoxy]-methyl-guanosine), buciclovir, famciclovir, ganciclovir(9-[2-hydroxy-1-(hydroxymethyl)ethoxyl-methyl]-guanosine), penciclovir, valciclovir, trifluorothymidine, AZT (3′-azido-3′-thymidine), AIU (5′-iodo-5′-amino-2′,5′-dideoxyuridine), ara-A (adenosine-arabinoside; Vivarabine), ara-C (cytidine-arabinoside), ara-G (9-beta-D-arabinofuranosylguanine), ara-T, 1-beta-D-arabinofuranosyl thymine, 5-ethyl-2′-deoxyuridine, 5-iodo-5′-amino-2,5′-dideoxyuridine, 1-[2-deoxy-2-fluoro-beta-D-arabino furanosyl]-5-iodouracil, idoxuridine (5-iodo-2′deoxyuridine), fludarabine(2-Fluoroadenine 9-beta-D-Arabinofuranoside), gencitabine, 3′-deoxyadenosine (3-dA), 2′,3′-dideoxyinosine (ddI), 2′,3′-dideoxycytidine (ddC), 2′,3′-dideoxythymidine (ddT), 2′,3′-dideoxyadenosine (ddA), 2,3′-dideoxyguanosine (ddG), 2-chloro-2′-deoxyadenosine (2CdA), 5-fluorodeoxyuridine, BVaraU ((E)-5-(2-bromovinyl)-1-beta-D-arabinofuranosyluracil), BVDU (5-bromovinyl-deoxyuridine), FIAU (1-(2-deoxy-2-fluoro-beta-D-arabinofuranosyl)-5-iodouracil), 3TC (2′-deoxy-3′-thiacytidine), dFdC gemcitabine(2′,2′-difluorodeoxycytidine), dFdG (2′,2′-difluorodeoxyguanosine), 5-fluorodeoxyuridine (FdUrd), d4T (2′,3′didehydro-3′-deoxythymidine), ara-M (6-methoxy purinearabinonucleoside), IudR (5-Jodo-2′deoxyuridine), CaFdA (2-chloro-2-ara-fluoro-deoxyadenosine), ara-U (1-beta-D-arabinofuranosyluracil), FBVAU (E)-5-(2-bromovinyl)-1-(2-deoxy-2-fluoro-beta-D-arabinofuranosyl)uracil, FMAU 1-(2-deoxzy-2-fluoro-beta-D-arabinofuranosyl)-5-methyluracil, FLT 3′-fluoro-2′-deoxythymidine, 5-Br-dUrd 5-bromodeoxyuridine, 5-Cl-dUrd 5-chlorodeoxyuridine, dFdU 2′,2′-difluorodeoxyuridine, (−)Carbovir (C-D4G), 2,6-Diamino-ddP (ddDAPR; DAPDDR; 2,6-Diamino-2′,3′-dideoxypurine-9-ribofuranoside), 9-(2′-Azido-2′,3′-dideoxy-β-D-erythropentofuranosyl)adenine(2′-Azido-2′,3′-dideoxyadenosine; 2′-N3ddA), 2′FddT (2′-Fluoro-2′,3′-dideoxy-β-D-erythro-pentofuranosyl)thymine), 2′-N3ddA(β-D-threo) (9-(2′-Azido-2′,3′-dideoxy-β-D-threopentofuranosyl)adenine), 3-(3-Oxo-1-propenyl)AZT (3-(3-Oxo-1-propenyl)-3′-azido-3′-deoxythymidine), 3′-Az-5-Cl-ddC (3′-Azido-2′,3′-dideoxy-5-chlorocytidine), 3′-N3-3′-dT (3′-Azido-3′-deoxy-6-azathymidine), 3′-F-4-Thio-ddT (2′,3′-Dideoxy-3′-fluoro-4-thiothymidine), 3′-F-5-Cl-ddC (2′,3′-Dideoxy-3′-fluoro-5-chlorocytidine), 3′-FddA (B-D-Erythro) (9-(3′-Fluoro-2′,3′-dideoxy-B-D-erythropentafuranosyl)adenine), Uravidine (3′-Azido-2′,3′-dideoxyuridine; AzdU), 3′-FddC (3′-Fluoro-2′,3′-dideoxycytidine), 3′(2,6-Diaminopurine-3′-fluoro-2′,3′-dideoxyriboside), 3′-FddG (3′-Fluoro-2′,3′-dideoxyguanosine, 3′-FddU (3′-Fluoro-2′,3′-dideoxyuridine), 3′-Hydroxymethyl-ddC (2′,3′-Dideoxy-3′-hydroxymethyl cytidine; BEA-005), 3′-N3-5-CF3-ddU (3′-Azido-2′,3′-dideoxy-5-trifluoromethyluridine), 3′-N3-5-Cyanomethyloxy-ddU (3′-Azido-2′,3′-dideoxy-5-[(cyanomethyl)oxy]uridine), 3′-N3-5-F-ddC (3′-Azido-2′,3′-dideoxy-5-fluorocytidine), 3′-N3-5-Me-ddC (CS-92; 3′-Azido-2′,3′-dideoxy-5-methylcytidine), 3′-N3-5-NH2-ddU (3′-Azido-2′,3′-dideoxy-5-aminouridine), 3′-N3-5-NHMe-ddU (3′-Azido-2′,3′-dideoxy-5-methyaminouridine), 3′-N3-5-NMe2-ddU (3′-Azido-2′,3′-dideoxy-5-dimethylaminouridine), 3′-N3-5-OH-ddU (3′-Azido-2′,3′-dideoxy-5-hydroxyuridine), 3′-N3-5-SCN-ddU (3′-Azido-2′,3′-dideoxy-5-thiocyanatouridine), 3′-N3-ddA (9-(3′-Azido-2′,3′-dideoxy-B-D-erythropentafuranosyl)adenine), 3′-N3-ddC (CS-91; 3′-Azido-2′,3′-dideoxycytidine), 3′-N3ddG (AZG; 3′-Azido-2′,3′-dideoxyguanosine), 3′-N3-N4-5-diMe-ddC (3′-Azido-2′,3′-dideoxy-N4-5-dimethylcytidine), 3′-N3-N4-OH-5-Me-ddC (3′-Azido-2′,3′-dideoxy-N4-OH-5-methylcytidine), 4′-Az-3′-dT (4′-Azido-3′-deoxythymidine), 4′-Az-5CldU (4′-Azido-5-chloro-2′-deoxyuridine), 4′-AzdA (4′-Azido-2′-deoxyadenosine), 4′-AzdC (4′-Azido-2′-deoxycytidine), 4′-AzdG (4′-Azido-2′-deoxyguanosine), 4′-AzdI (4′-Azido-2′-deoxyinosine), 4′-AzdU (4′-Azido-2′-deoxyuridine), 4′-Azidothymidine (4′-Azido-2′-deoxy-.beta.-D-erythro-pentofuranosyl-5-methyl-2,4-dioxopyrimidine), 4′-CN-T (4′-Cyanothymidine), 5-Et-ddC (2′,3′-Dideoxy-5-ethylcytidine), 5-F-ddC (5-Fluoro-2′,3′-dideoxycytidine), 6Cl-ddP (D2ClP; 6-Chloro-ddP; CPDDR; 6-Chloro-9-(2,3-dideoxy-.beta.-D-glyceropentofuranosyl)-9H-purine), 935U83 (2′,3′-Dideoxy-3′-fluoro-5-chlorouridine; 5-Chloro-2′,3′-dideoxy-3′-fluorouridine; FddClU; Raluridine), AZddBrU (3′-N3-5-Br-ddU; 3′-Azido-2′,3′-dideoxy-5-bromouridine), AzddClU; AzddClUrd (3′-Azido-5-chloro-2′,3′-dideoxyuridine), AZddEtU (3′-N3-5-EtddU; CS-85; 3′-Azido-2′,3′-dideoxy-5-ethyluridine), AZddFU (3′-Azido-2′,3′-dideoxy-5-fluorouridine), AZddIU (3′-N3-5-I-ddU; 3′-Azido-2′,3′-dideoxy-5-iodouridine), AZT-2,5′-anhydro (2,5′-Anhydro-3′-azido-3′-deoxythymidine), AZT-α-L (α-L-AZT), AZU-2,5′-anhydro (2,5′-Anhydro-3′-azido-2′,3′-dideoxyuridine), C-analog of 3′-N3-ddU (3′-Azido-2′,3′-dideoxy-5-aza-6-deazauridine), D2SMeP (9-(2,3-Dideoxy-β-D-ribofuranosyl)-6-(methylthio)purine), D4A (2′,3′-Dideoxydidehydroadenosine), D4C (2′,3′-Didehydro-3′-deoxycytidine), D4DAP (2,6-Diaminopurine-2′,3′-dideoxydidehydroriboside; ddeDAPR), D4FC (D-D4FC; 2′,3′-Didehydro-2′,3′-dideoxy-5-fluorocytidine), D4G (2′,3′-Didehydro-2′,3′-dideoxyguanosine), DMAPDDR (N-6-dimethyl ddA; 6-Dimethylaminopurine-2′,3′-dideoxyriboside), dOTC (−) ((−)-2′-Deoxy-3′-oxa-4′-thiocytidine), dOTC (+) ((+)-2′-Deoxy-3′-oxa-4′-thiocytidine), dOTFC (−) ((−)-2′-Deoxy-3′-oxa-4′-thio-5-fluorocytidine), dOTFC (+) ((+)-2′-Deoxy-3′-oxa-4′-thio-5-fluorocytidine), DXG ((−)-β-Dioxolane-G), DXC-α-L-(α-L-Dioxalane-C), FddBrU (2′,3′-Dideoxy-3′-fluoro-5-bromouridine), FddIU (3′-Fluoro-2′,3′-dideoxy-5-iodouridine), FddT (Alovudine; 3′-FddT; FddThD; 3′-FLT; FLT), FTC (Emtricitabine; Coviracil; (−)-FTC; (−)-2′,3′-Dideoxy-5-fluoro-3′-thiacytidine), FTC-α-L-(α-L-FTC), L-D4A (L-2′,3′-Didehydro-2′,3′-dideoxyadenosine), L-D4FC (L-2′,3′-Didehydro-2′,3′-dideoxy-5-fluorocytidine), L-D4I (L-2′,3′-Didehydro-2′,3′-dideoxyinosine), L-D4G (L-2′,3′-Didehydro-2′,3′-deoxyguanosine), L-FddC (β-L-5F-ddC), Lodenosine (F-ddA; 2′-FddA (B-D-threo); 2′-F-dd-ara-A; 9-(2′-Fluoro-2′,3′-dideoxy-B-D-threopenta-furanosyl)adenine), MeAZddIsoC (5-Methyl-3′-azido-2′,3′-dideoxyisocytidine), N6-Et-ddA (N-Ethyl-2′,3′-dideoxyadenosine), N-6-methyl ddA (N6-Methyl-2′,3′-dideoxyadenosine) or RO31-6840 (1-(2′,3′-Dideoxy-2′-fluoro-β-D-threo-pentofuranosyl)cytosine).

27. The composition according to claim 1, wherein the nucleoside analog is cytidine analog, a guanosine analog or an adenosine selected from the group consisting of dFdC gemcitabine (2′,2′-difluorodeoxycytidine), 2-chloro-2′-deoxyadenosine (2CdA), CaFdA (2-chloro-2-ara-fluoro-deoxyadenosine), fludarabine (2-Fluoroadenine 9-beta-D-Arabinofuranoside), 2′,3 ′-dideoxycytidine (ddC), 2′,3′-dideoxyadenosine (ddA), 2′,3′-dideoxyguanosine (ddG), ara-A (adenosine-arabinoside; Vivarabine), ara-C (cytidine-arabinoside), ara-G (9-beta-D-arabinofuranosylguanine), aciclovir (9-[2-hydroxy-ethoxy]-methyl-guanosine), buciclovir, famciclovir, ganciclovir (9-[2-hydroxy-1-(hydroxymethyl)ethoxyl-methyl]-guanosine), penciclovir, valciclovir, 3TC (2′-deoxy-3′-thiacytidine), dFdG (2′,2′-difluorodeoxyguanosine), 2,6-Diamino-ddP (ddDAPR; DAPDDR; 2,6-Diamino-2′,3′-dideoxypurine-9-ribofuranoside), 9-(2′-Azido-2′,3′-dideoxy-β-D-erythropentofuranosyl)adenine (2′-Azido-2′,3′-dideoxyadenosine; 2′-N3ddA), 2′-N3ddA(β-D-threo) (9-(2′-Azido-2′,3′-dideoxy-β-D-threopentofuranosyl)adenine), 3′-Az-5-Cl-ddC (3′-Azido-2′,3′-dideoxy-5-chlorocytidine), 3′-F-5-Cl-ddC (2′,3′-Dideoxy-3′-fluoro-5-chlorocytidine), 3′-FddA (B-D-Erythro) (9-(3′-Fluoro-2′,3′-dideoxy-B-D-erythropentafuranosyl)adenine), 3′-FddC (3′-Fluoro-2′,3′-dideoxycytidine), 3′-F-ddDAPR (2,6-Diaminopurine-3′-fluoro-2′,3′-dideoxyriboside), 3′-FddG (3′-Fluoro-2′,3′-dideoxyguanosine), 3′-Hydroxymethyl-ddC (2′,3′-Dideoxy-3′-hydroxymethyl cytidine; BEA-005), 3′-N3-5-F-ddC (3′-Azido-2′,3′-dideoxy-5-fluorocytidine), 3′-N3-5-Me-ddC (CS-92; 3′-Azido-2′,3′-dideoxy-5-methylcytidine), 3′-N3-ddA (9-(3′-Azido-2′,3′-dideoxy-B-D-erythropentafuranosyl)adenine), 3′-N3-ddC (CS-91; 3′-Azido-2′,3′-dideoxycytidine), 3′-N3ddG (AZG; 3′-Azido-2′,3′-dideoxyguanosine), 3′-N3-N4-5-diMe-ddC (3′-Azido-2′,3′-dideoxy-N4-5-dimethylcytidine), 3′-N3-N4-OH-5-Me-ddC (3′-Azido-2′,3′-dideoxy-N4-OH-5-methylcytidine), 4′-AzdA (4′-Azido-2′-deoxyadenosine), 4′-AzdC (4′-Azido-2′-deoxycytidine), 4′-AzdG (4′-Azido-2′-deoxyguanosine), 5-Et-ddC (2′,3′-Dideoxy-5-ethylcytidine), 5-F-ddC (5-Fluoro-2′,3′-dideoxycytidine), 6Cl-ddP (D2ClP; 6-Chloro-ddP; CPDDR; 6-Chloro-9-(2,3-dideoxy-.beta.-D-glyceropentofuranosyl)-9H-purine), D2SMeP (9-(2,3-Dideoxy-β-D-ribofuranosyl)-6-(methylthio)purine), D4A (2′,3′-Dideoxydidehydroadenosine), D4C (2′,3′-Didehydro-3′-deoxycytidine), D4DAP (2,6-Diaminopurine-2′,3′-dideoxydidehydroriboside; ddeDAPR), D4FC (D-D4FC; 2′,3′-Didehydro-2′,3′-dideoxy-5-fluorocytidine), D4G (2′,3′-Didehydro-2′,3′-dideoxyguanosine), DMAPDDR (N-6-dimethyl ddA; 6-Dimethylaminopurine-2′,3′-dideoxyriboside), dOTC (−) ((−)-2′-Deoxy-3′-oxa-4′-thiocytidine), dOTC (+) ((+)-2′-Deoxy-3′-oxa-4′-thiocytidine), dOTFC (−) ((−)-2′-Deoxy-3′-oxa-4′-thio-5-fluorocytidine), dOTFC (+) ((+)-2′-Deoxy-3′-oxa-4′-thio-5-fluorocytidine), DXG ((−)-β-Dioxolane-G), DXC-α-L-(α-L-Dioxalane-C), FTC (Emtricitabine; Coviracil; (−)-FTC; (−)-2′,3′-Dideoxy-5-fluoro-3′-thiacytidine), FTC-α-L-(α-L-FTC), L-D4A (L-2′,3′-Didehydro-2′,3′-dideoxyadenosine), L-D4FC (L-2′,3′-Didehydro-2′,3′-dideoxy-5-fluorocytidine), L-D41 (L-2′,3′-Didehydro-2′,3′-dideoxyinosine), L-D4G (L-2′,3′-Didehydro-2′,3′-deoxyguanosine), L-FddC (β-L-5F-ddC), Lodenosine (F-ddA; 2′-FddA (B-D-threo); 2′-F-dd-ara-A; 9-(2′-Fluoro-2′,3′-dideoxy-B-D-threopentafuranosyl)adenine), MeAZddIsoC (5-Methyl-3′-azido-2′,3′-dideoxyisocytidine), N6-Et-ddA (N-Ethyl-2′,3′-dideoxyadenosine), N-6-methyl ddA (N6-Methyl-2′,3′-dideoxyadenosine) or RO31-6840 (1-(2′,3 ′-Dideoxy-2′-fluoro-β-D-threo-pentofuranosyl)cytosine).

28. The composition according to claim 1, wherein the at least one nucleoside analogue is selected from the group consisting of D4T, ddC, AZT, ACV, 3TC, ddA Fludarabine, Cladribine, araC, gemcitabine, Clofarabine, Nelarabine (araG), and Ribavirin.

29. The composition according to claim 1, wherein the at least one nucleoside analogue is gemcitabine or AZT.

30. The composition according to claim 1, comprising at least two nucleoside analogues, such as at least 3 nucleoside analogues, for example at least 4 nucleoside analogues, such as at least 5 nucleoside analogues.

31. The composition according to claim 1, comprising at least three compounds capable of enhancing gap-junction communication, such as at least 3 compounds, for example at least 4 compounds, such as at least 5 compounds.

32. A method of treating cancer comprising administering to a patient in need thereof a therapeutically effective amount of at least one compound capable of increasing gap-junction communication, and at least one nucleoside analogue.

33. The method of claim 32, further comprising administering a source of deoxyribonucleoside kinase.

34. The method of claim 32, wherein the cancer is a multicellular cancer type.

35. The method of claim 33, wherein the cancer is selected from the group consisting of glioblastoma, bladder cancer, neuroblastoma, esophageal cancer, tongue cancer, hepatocellular carcinoma, lung cancer, malignant melanoma, ovarian cancer, prostate cancer, renal cell carcinoma, and breast cancer.

36. The method of claim 35, wherein the cancer is breast cancer or glioblastoma.

37. The method of claim 33, wherein the deoxyribonucleoside kinase is administered to the cancer cells using a gene therapy vector.

38. The method of claim 37, wherein the gene therapy vector is a virus vector selected from the group consisting of Herpes simplex viral vector, adenoviral vector, adenovirus-associated viral vector, lentiviral vector, retroviral vector, and a vacciniaviral vector.

39. The method of claim 38, wherein the gene therapy vector is administered to the cancer cells by implanting a composition of packaging cells capable of producing an infective virion comprising the viral vector.

40. The method of claim 39, wherein the packaging cells are encapsulated, and/or wherein the packaging cells are attached to a support matrix.

41. The method of claim 37, wherein the gene therapy vector is a plasmid vector.

42. The method of claim 41, wherein the plasmid vector is selected from the group consisting of general eukaryotic expression vectors, vectors for stable and transient expression and epitag vectors as well as their TOPO derivatives for fast cloning of desired inserts.

43. The method of claim 33, wherein the thymidine kinase is administered to the cancer cells by implanting a composition of human stem or precursor/progenitor cells, comprising a heterologous expression construct capable of expressing said deoxyribonucleoside kinase, and wherein said human stem cells are capable of forming a tight junction with cells in the tumour.

44. The method of claim 43, wherein said human stem or precursor/progenitor cells are human neural stem or precursor/progenitor cells.

45. The method of claim 32, comprising administration of the composition of claim 1.

46. The method of claim 32, wherein said compound capable of enhancing gap-junction communication is 4-phenylbutyrate, or a pharmaceutically acceptable salt thereof.

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. A method of augmenting the therapeutic activity of a nucleoside analogue based cancer therapy, said method comprising administering to a patient an amount of at least one compound capable of enhancing gap-junction communication and thereby augmenting the therapeutic activity of said nucleoside analogue based therapy.

59. The method of claim 58, wherein said nucleoside analogue based therapy further comprises administration of a source of deoxyribonucleoside kinase.

60. The method of claim 58, wherein said compound capable of enhancing gap-junction communication is 4-phenylbutyrate or a pharmaceutically acceptable salt thereof.

61. The method of claim 58, comprising administering the composition according to claim 1.

62. Pharmaceutical articles containing at least one nucleoside analogue and at least one compound capable of enhancing gap-junction communication as a combination for the simultaneous, separate or successive administration in cancer therapy.

63. Articles of claim 62, further comprising a source of deoxyribonucleoside kinase.

64. Articles of claim 62, wherein said compound capable of enhancing gap-junction communication is 4-phenylbutyrate or a pharmaceutically acceptable salt thereof.

65. Articles of claim 62, comprising the composition according to claim 1.

66. Articles of claim 62, wherein the cancer is a multicellular cancer type.

67. Articles of claim 62, wherein the cancer is selected from the group consisting of glioblastoma, bladder cancer, neuroblastoma, esophageal cancer, tongue cancer, hepatocellular carcinoma, lung cancer, malignant melanoma, ovarian cancer, prostate cancer, renal cell carcinoma, and breast cancer.

68. Articles of claim 62, wherein the cancer is breast cancer or glioblastoma.
